SEQUENCE LISTING (38 sequences)

SEQ ID NO: 1 - protein, 486 aa, Tilapia nilotica
MVLEMLNPMHYKVTSMVSEVVPFASIAVLLLTGFLLLVWNYKNTSSIPGPGYFLGIGPLISYLRFLWMGIGSACNYYNKTYGEFIRVWIGGEETLIISKSSSVFHVMKHSHYTSRFGSKPGLQFIGMHEKGIIFNNNPVLWKAVRTYFMKALSGPGLVRMVTVCADSITKHLDKLEEVRNDLGYVDVLTLMRRIMLDTSNNLFLGIPLDEKAIVCKIQGYFDAWQALLLKPDIFFKIPWLYRKYEKSVKDLKEDMEILIEKKRRRIFTAEKLEDCMDFATELILAEKRGELTKENVNQCILEMLIAAPDTMSVTVFFMLFLIAKHPQVEEELMKEIQTVVGERDIRNDDMQKLEVVENFIYESMRYQPVVDLVMRKALEDDVIDGYPVKKGTNIILNIGRMHRLEFFPKPNEFTLENFAKNVPYRYFQPFGFGPRACAGKYIAMVMMKVTLVILLRRFQVQTPQDRCVEKMQKKNDLSLHPDETSG

SEQ ID NO: 2 - protein, 486 aa, Oryzias latipes
MFLEMLNPMQYNVTIMVPETVTVSAMPLLLIMGLLLLIWNCESSSSIPGPGYCLGIGPLISHGRFLWMGIGSACNYYNKMYGEFMRVWISGEETLIISKSSSMFHVMKHSHYISRFGSKRGLQCIGMHENGIIFNNNPSLWRTIRPFFMKALTGPGLVRMVEVCVESIKQHLDRLGEVTDTSGYVDVLTLMRHIMLDTSNMLFLGIPLDESAIVKKIQGYFNAWQALLIKPNIFFKISWLYRKYERSVKDLKDEIAVLVEKKRHKVSTAEKLEDCMDFATDLIFAERRGDLTKENVNQCILEMLIAAPDTMSVTLYFMLLLVAEYPEVEAAILKEIHTVVGDRDIKIEDIQNLKVVENFINESMRYQPVVDLVMRRALEDDVIDGYPVKKGTNIILNIGRMHRLEYFPKPNEFTLENFEKNVPYRYFQPFGFGPRGCAGKYIAMVMMKVVLVTLLRRFQVKTLQKRCIENIPKKNDLSLHPNEDRH

SEQ ID NO: 3 - protein, 486 aa, Danio rerio
MILEMLNPMHYNLTSMVPEVMPVATLPILLLTGFLFFVWNHEETSSIPGPGYCMGIGPLISHLRFLWMGLGSACNYYNKMYGEFVRVWISGEETLVISKSSSTFHIMKHDHYSSRFGSTFGLQYMGMHENGVIFNNNPAVWKALRPFFVKALSGPSLARMVTVCVESVNNHLDRLDEVTNALGHVNVLTLMRRTMLDASNTLFLRIPLDEKNIVLKIQGYFDAWQALLIKPNIFFKISWLSRKHQKSIKELRDAVGILAEEKRHRIFTAEKLEDHVDFATDLILAEKRGELTKENVNQCILEMMIAAPDTLSVTVFFMLCLIAQHPKVEEALMKEIQTVLGERDLKNDDMQKLKVMENFINESMRYQPVVDIVMRKALEDDVIDGYPVKKGTNIILNIGRMHKLEFFPKPNEFTLENFEKNVPYRYFQPFGFGPRSCAGKFIAMVMMKVMLVSLLRRFHVKTLQGNCLENMQKTNDLALHPDESRS

SEQ ID NO: 4 - protein, 487 aa, Carassius auratus
VLELLMQGAHNSSYGAQDNVCGAMATLLLLLLCLLLAIRHHWTEKDHVPGPCFLLGLGPLLSYCRLIWSGIGTASNYYNSKYGDIVRVWINGEETLILSRSSAVYHVLRKSLYTSRFGSKLGLQCIGMHEQGIIFNSNVALWKKVRTFYAKALTGPGLQRTLEICITSTNTHLDNLSHLMDARGQVDILNLLRCIVVDISNRLFLGVPLNEHDLLQKIHKYFDTWQTVLIKPDVYFRLAWWLHGKHKRDAQELQDAIAALIEQKRVQLTRAEKFDQLDFTGELIFAQSHGELSTENVRQCVLEMIIAAPDTLSISLFFMLLLLKQNPDVELKILQEMNAVLAGRSLQHSHLSGLHILESFINESLRFHPVVDFTMRRALDDDVIEGYEVKKGTNIILNVGRMHRSEFFPKPNEFSLDNFQKNVPSRFFQPFGSGPRSCVGKHIAMVMMKSILVTLLSRFSVCPVKGCTVDSIPQTNDLSQQPVEEPS

SEQ ID NO: 5 - protein, 484 aa, Ictalurus punctatus
MEEVLKGTVNFAATVQVTLMALTGTLLLILLHRIFTAKNWRNQSGVPGPGWLLGLGPIMSYSRFLWMGIGSACNYYNEKYGSIARVWISGEETFILSKSSAVYHVLKSNNYTGRFASKKGLQCIGMFEQGIIFNSNMALWKKVRTYFTKALTGPGLQKSVDVCVSATNKQLNVLQEFTDHSGHVDVLNLLRCIVVDVSNRLFLRIPLNEKDLLIKIHRYFSTWQAVLIQPDVFFRLNFVYKKYHLAAKELQDEMGKLVEQKRQAINNMEKLDETDFATELIFAQNHDELSVDDVRQCVLEMVIAAPDTLSISLFFMLLLLKQNSVVEEQIVQEIQSQIGERDVESADLQKLNVLERFIKESLRFHPVVDFIMRRALEDDEIDGYRVAKGTNLILNIGRMHKSEFFQKPNEFNLENFENTVPSRYFQPFGCGPRACVGKHIAMVMTKAILVTLLSRFTVCPRHGCTVSTIKQTNNLSMQPVEEDP

SEQ ID NO: 6 - protein, 486 aa, Carassius auratus
VVDLLIQRAHNGTERAQDNACGATATILLLLLCLLLAIRHHRPHKSHIPGPSFFFGLGPVVSYCRFIWSGIGTASNYYNSKYGDIVRVWINGEETLILSRSSAVYHVLRKSLYTSRFGSKLGLQCIGMHEQGIIFNSNVALWKKVRAFYAKALTGPGLQRTMEICTTSTNSHLDDLSQLTDAQGQLDILNLLRCIVVDVSNRLFLGVPLNEHDLLQKIHKYFDTWQTVLIKPDVYFRLDWLHRKHKRDAQELQDAITALIEQKKVQLAHAEKLDHLDFTAELIFAQSHGELSAENVRQCVLEMVIAAPDTLSISLFFMLLLLKQNPDVELKILQEMDSVLAGQSLQHSHLSKLQILESFINESLRFHPVVDFTMRRALDDDVIEGYNVKKGTNIILNVGRMHRSEFFSKPNQFSLDNFHKNVPSRFFQPFGSGPRSCVGKHIAMVMMKSILVALLSRFSVCPMKACTVENIPQTNNLSQQPVEEPS

SEQ ID NO: 7 - protein, 486 aa, Sus scrofa (pig) placental
MVLEMLNPMYYKITSMVSEVVPFASIAVLLLTGFLLLLWNYENTSSIPSPGYFLGIGPLISHFRFLWMGIGSACNYYNEMYGEFMRVWIGGEETLIISKSSSVFHVMKHSHYTSRFGSKPGLECIGMYEKGIIFNNDPALWKAVRTYFMKALSGPGLVRMVTVCADSITKHLDKLEEVRNDLGYVDVLTLMRRIMLDTSNNLFLGIPLDEKAIVCKIQGYFDAWQALLLKPEFFFKFSWLYKKHKESVKDLKENMEILIEKKRCSIITAEKLEDCMDFATELILAEKRGELTKENVNQCILEMLIAAPDTLSVTVFFMLFLIAKHPQVEEAIVKEIQTVIGERDIRNDDMQKLKVVENFIYESMRYQPVVDLVMRKALEDDVIDGYPVKKGTNIILNIGRMHRLEFFPKPNEFTLENFAKNVPYRYFQPFGFGPRACAGKYIAMVMMKVTLVILLRRFQVQTPQDRCVEKMQKKNDLSLHPDETSG

SEQ ID NO: 8 - protein, 476 aa, Sus scrofa (pig) embryo
LVSIAPNTTVGLPSGIPMATRSLILLVCLLLMVWSHSEKKTIPGPSFCLGLGPLMSYLRFIWTGIGTASNYYNNKYGDIVRVWINGEETLILSRASAVHHVLKNRKYTSRFGSKQGLSCIGMNEKGIIFNNNVALWKKIRTYFTKALTGPNLQQTVEVCVTSTQTHLDNLSSLSYVDVLGFLRCTVVDISNRLFLGVPVDEKELLQKIHKYFDTWQTVLIKPDIYFKFSWIHQRHKTAAQELQDAIESLVERKRKEMEQAEKLDNINFTAELIFAQGHGELSAENVRQCVLEMVIAAPDTLSISLFFMLLLLKQNPHVELQLLQEIDTIVGDSQLQNQDLQKLQVLESFINECLRFHPVVDFTMRRALFDDIIDGHRVQKGTNIILNTGRMHRTEFFHKANEFSLENFQKNTPRRYFQPFGSGPRACVGRHIAMVMMKSILVTLLSQYSVCPHEGLTLDCLPQTNNLSQQPVEHHQ

SEQ ID NO: 9 - protein, 484 aa, Sus scrofa (pig) ovary
MVLEMLNPMNISSMVSEAVLFGSIAILLLIGLLLWVWNYEDTSSIPGPGYFLGIGPLISHFRFLWMGIGSACNYYNKMYGEFMRVWIGGEETLIISKSSSIFHIMKHNHYTCRFGSKLGLECIGMHEKGIMFNNNPALWKAVRPFFTKALSGPGLVRMVTVCADSITKHLDKLEEVRNDLGYVDVLTLMRRIMLDTSNNLFLGIPLDESALVHKVQGYFDAWQALLLKPDIFFKISWLYRKYEKSVKDLKDAMEILIEEKRHRISTAEKLEDSMDFTTQLIFAEKRGELTKENVNQCVLEMMIAAPDTMSITVFFMLFLIANHPQVEEELMKEIYTVVGERDIRNDDMQKLKVVENFIYESMRYQPVVDFVMRKALEDDVIDGYPVKKGTNIILNIGRMHRLEFFPKPNEFTLENFAKNVPYRYFQPFGFGPRACAGKYIAMVMMKVILVTLLRRFQVQTQQGQCVEKMQKKNDLSLHPHETSG

SEQ ID NO: 10 - protein, 486 aa, Bos taurus
MVLEMLNPMHFNITTMVPAAMPAATMPILLLTCLLLLIWNYEGTSSIPGPGYCMGIGPLISYARFLWMGIGSACNYYNKMYGEFIRVWICGEETLIISKSSSMFHVMKHSHYVSRFGSKPGLQCIGMHENGIIFNNNPALWKVVRPFFMKALTGPGLVQMVAICVGSIGRHLDKLEEVTTRSGCVDVLTLMRRIMLDTSNTLFLGIPMDESAIVVKIQGYFDAWQALLLKPNIFFKISWLYKKYEKSVKDLKDAIDILVEKKRRRISTAEKLEDHMDFATNLIFAEKRGDLTRENVNQCVLEMLIAAPDTMSVSVFFMLFLIAKHPSVEEAIMEEIQTVVGERDIRIDDIQKLKVVENFIYESMRYQPVVDLVMRKALEDDVIDGYPVKKGTNIILNIGRMHRLEFFPKPNEFTLENFAKNVPYRYFQPFGFGPRGCAGKYIAMVMMKVILVTLLRRFQVKALQGRSVENIQKKNDLSLHPDETSD

SEQ ID NO: 11 - protein, 485 aa, Equus caballus
VMEILLREARNGTDPRYENPRGITLLLLLCLVLLLTVWNRHEKKCSIPGPSFCLGLGPLMSYCRFIWMGIGTASNYYNEKYGDMVRVWISGEETLVLSRPSAVYHVLKHSQYTSRFGSKLGLQCIGMHEQGIIFNSNVTLWRKVRTYFAKALTGPGLQRTLEICTMSTNTHLDGLSRLTDAQGHVDVLNLLRCIVVDISNRLFLDVPLNEQNLLFKIHRYFETWQTVLIKPDFYFRLKWLHDKHRNAAQELHDAIEDLIEQKRTELQQAEKLDNLNFTEELIFAQSHGELTAENVRQCVLEMVIAAPDTLSISVFFMLLLLKQNAEVERRILTEIHTVLGDTELQHSHLSQLHVLECFINEALRFHPVVDFSYRRALDDDVIEGFRVPRGTNIILNVGRMHRSEFYPKPADFSLDNFNKPVPSRFFQPFGSGPRSCVGKHIAMVMMKAVLLMVLSRFSVCPEESCTVENIAHTNDLSQQPVEDKH

SEQ ID NO: 12 - protein, 486 aa, Mus musculus
MVLETLNPLHYNITSLVPDTMPVATVPILILMCFLFLIWNHEETSSIPGPGYCMGIGPLISHGRFLWMGVGNACNYYNKTYGDFVRVWISGEETFIISKSSSVSHVMKHWHYVSRFGSKLGLQCIGMYENGIIFNNNPAHWKEIRPFFTKALSGPGLVRMIAICVESTTEHLDRLQEVTTELGNINALNLMRRIMLDTSNKLFLGVPLDENAIVLKIQNYFDAWQALLLKPDIFFKISWLCKKYKDAVKDLKGAMEILIEQKRQKLSTVEKLDEHMDFASQLIFAQNRGDLTAENVNQCVLEMMIAAPDTLSVTLFFMLILIAEHPTVEEEMMREIETVVGDRDIQSDDMPNLKIVENFIYESMRYQPVVDLIMRKALQDDVIDGYPVKKGTNIILNIGRMHKLEFFPKPNEFSLENFEKNVPSRYFQPFGFGPRSCVGKFIAMVMMKAILVTLLRRCRVQTMKGRGLNNIQKNNDLSMHPIERQP

SEQ ID NO: 13 - protein, 478 aa, Rattus norvegicus
VVARSLCDLKCHPIDGISMATRTLILLVCLLLVAWSHTDKKIVPGPSFCLGLGPLLSYLRFIWTGIGTASNYYNNKYGDIVRVWINGEETLILSRSSAVHHVLKNGNYTSRFGSIQGLSYLGMNERGIIFNNNVTLWKKIRTYFAKALTGPNLQQTVDVCVSSIQAHLDHLDSLGHVDVLNLLRCTVLDISNRLFLNVPLNEKELMLKIQKYFHTWQDVLIKPDIYFKFRWIHHRHKTATQELQDAIKRLVDQKRKNMEQADKLDNINFTAELIFAQNHGELSAENVTQCVLEMVIAAPDTLSLSLFFMLLLLKQNPHVEPQLLQEIDAVVGERQLQNQDLHKLQVMESFIYECLSFHPVVDFTMRRALSDDIIEGYRISKGTNIILNTGRMHRTEFFLKGNQFNLEHFENNVPRPPTFQPFGSGPRACIGKHMAMVMMKSILVTLLSQYSVCTHEGPILDCLPQTNNLSQQPVEHQQ

SEQ ID NO: 14 - protein, 486 aa, Oryctolagus cuniculus
MLLEVLNPRHYNVTSMVSEVVPIASIAILLLTGFLLLVWNYEDTSSIPGPSYFLGIGPLISHCRFLWMGIGSACNYYNKMYGEFMRVWVCGEETLIISKSSSMFHVMKHSHYISRFGSKLGLQFIGMHEKGIIFNNNPALWKAVRPFFTKALSGPGLVRMVTICADSITKHLDRLEEVCNDLGYVDVLTLMRRIMLDTSNMLFLGIPLDESAIVVNIQGYFDAWQALLLKPDIFFKISWLCRKYEKSVKDLKDAMEILIAEKRHRISTAEKLEDSIDFATELIFAEKRGELTRENVNQCILEMLIAAPDTMSVSVFFMLFLIAKHPQVEEAIIREIQTVVGERDIRIDDMQKLKVVENFINESMRYQPVVDLVMRKALEDDVIDGYPVKKGTNIILNLGRMHRLEFFPKPNEFTLENFAKNVPYRYFQPFGFGPRGCAGKYIAMVMMKVVLVTLLRRFHVQTLQGRCVEKMQKKNDLSLHPDETRD

SEQ ID NO: 15 - protein, 486 aa, Homo sapiens
MVLEMLNPIHYNITSIVPEAMPAATMPVLLLTGLFLLVWNYEGTSSIPGPGYCMGIGPLISHGRFLWMGIGSACNYYNRVYGEFMRVWISGEETLIISKSSSMFHIMKHNHYSSRFGSKLGLQCIGMHEKGIIFNNNPELWKTTRPFFMKALSGPGLVRMVTVCAESLKTHLDRLEEVTNESGYVDVLTLLRRVMLDTSNTLFLRIPLDESAIVVKIQGYFDAWQALLIKPDIFFKISWLYKKYEKSVKDLKDAIEVLIAEKRRRISTEEKLEECMDFATELILAEKRGDLTRENVNQCILEMLIAAPDTMSVSLFFMLFLIAKHPNVEEAIIKEIQTVIGERDIKIDDIQKLKVMENFIYESMRYQPVVDLVMRKALEDDVIDGYPVKKGTNIILNIGRMHRLEFFPKPNEFTLENFAKNVPYRYFQPFGFGPRGCAGKYIAMVMMKAILVTLLRRFHVKTLQGQCVESIQKIHDLSLHPDETKN

SEQ ID NO: 16 - protein, 466 aa, Gallus gallus
MPVATVPIIILICFLFLIWNHEETSSIPGPGYCMGIGPLISHGRFLWMGVGNACNYYNKTYGEFVRVWISGEETFIISKSSSVFHVMKHWNYVSRFGSKLGLQCIGMYENGIIFNNNPAHWKEIRPFFTKALSGPGLVRMIAICVESTIVHLDKLEEVTTEVGNVNVLNLMRRIMLDTSNKLFLGVPLDESAIVLKIQNYFDAWQALLLKPDIFFKISWLCKKYEEAAKDLKGAMEILIEQKRQKLSTVEKLDEHMDFASQLIFAQNRGDLTAENVNQCVLEMMIAAPDTLSVTLFIMLILIADDPTVEEKMMREIETVMGDREVQSDDMPNLKIVENFIYESMRYQPVVDLIMRKALQDDVIDGYPVKKGTNIILNIGRMHKLEFFPKPNEFSLENFEKNVPSRYFQPFGFGPRGCVGKFIAMVMMKAILVTLLRRCRVQTMKGRGLNNIQKNNDLSMHPIERQP

SEQ ID NO: 17 - protein, 486 aa, Poephila guttata
MFLEMLNPMHYNVTIMVPETVPVSAMPLLLIMGLLLLIRNCESSSSIPGPGYCLGIGPLISHGRFLWMGIGSACNYYNKMYGEFMRVWISGEETLIISKSSSMVHVMKHSNYISRFGSKRGLQCIGMHENGIIFNNNPSLWRTVRPFFMKALTGPGLIRMVEVCVESIKQHLDRLGDVTDNSGYVDVVTLMRHIMLDTSNTLFLGIPLDESSIVKKIQGYFNAWQALLIKPNIFFKISWLYRKYERSVKDLKDEIEILVEKKRQKVSSAEKLEDCMDFATDLIFAERRGDLTKENVNQCILEMLIAAPDTMSVTLYVMLLLIAEYPEVETAILKEIHTVVGDRDIRIGDVQNLKVVENFINESLRYQPVVDLVMRRALEDDVIDGYPVKKGTNIILNIGRMHRLEYFPKPNEFTLENFEKNVPYRYFQPFGFGPRSCAGKYIAMVMMKVVLVTLLKRFHVKTLQKRCIENMPKNNDLSLHLDEDSP

SEQ ID NO: 18 - protein, 50 aa, Homo sapiens
RNVIQISNDLENLRDLLHVLAFSKSCHLPWASGLETLDSLGGVLEASGYS

SEQ ID NO: 19 - protein, 50 aa, Pan troglodytes
RNMIQISNDLENLRDLLHVLAFSKSCHLPWASGLETLDSLGGVLEASGYS

SEQ ID NO: 20 - protein, 50 aa, Gorilla gorilla; identical to SEQ ID NO: 19

SEQ ID NO: 21 - protein, 50 aa, Orangutan
RNVIQISNDLENLRDLLHVLAFSKSCHLPWASGLETLDRLGGVLEASGYS

SEQ ID NO: 22 - protein, 50 aa, Rhesus monkey
RNVIQISNDLENLRDLLHLLAFSKSCHLPLASGLETLESLGDVLEASLYS

SEQ ID NO: 23 - protein, 50 aa, Rattus norvegicus
QNVLQIAHDLENLRDLLHLLAFSKSCSLPQTRGLQKPESLDGVLEASLYS

SEQ ID NO: 24 - protein, 50 aa, Rattus norvegicus; identical to SEQ ID NO: 23

SEQ ID NO: 25 - protein, 50 aa, Mus musculus
QNVLQIANDLENLRDLLHLLAFSKSCSLPQTSGLQKPESLDGVLEASLYS

SEQ ID NO: 26 - protein, 50 aa, Artificial Sequence
  reconstructed ancestral sequence
RNVIQISNDLENLRDLLHLLASSKSCPLPQARGLETLESLGGVLEASLYS

SEQ ID NO: 27 - protein, 50 aa, Sus scrofa
RNVIQISNDLENLRDLLHLLASSKSCPLPQARALETLESLGGVLEASLYS

SEQ ID NO: 28 - protein, 50 aa, Ovis
RNVIQISNDLENLRDLLHLLAASKSCPLPQVRALESLESLGVVLEASLYS

SEQ ID NO: 29 - protein, 50 aa, Bos taurus
RNVVQISNDLENLRDLLHLLAASKSCPLPQVRALESLESLGVVLEASLYS

SEQ ID NO: 30 - protein, 50 aa, Dog
RNVVQISNDLENLRDLLHLLASSKSCPLPRARGLETFESLGGVLEASLYS

SEQ ID NO: 31 - protein, 28 aa, Sus scrofa
NHYTCRFGSKLGLECIGMHEKGIMFNNN

SEQ ID NO: 32 - protein, 28 aa, Sus scrofa
SHYTSRFGSKPGLQFIGMHEKGIIFNNN

SEQ ID NO: 33 - protein, 28 aa, Sus scrofa
SHYTSRFGSKPGLECIGMYEKGIIFNND

SEQ ID NO: 34 - protein, 28 aa, White lipped peccary
SHYTSRFGSKPGLQFIGMHEKGIIFNNN

SEQ ID NO: 35 - DNA, 84 bp, Sus scrofa
caatcattacacgtgccgatttggcagcaaacttgggttggaatgcattggcatgcatgaaaaaggcatcatgtttaacaataa

SEQ ID NO: 36 - DNA, 84 bp, Sus scrofa
tagtcactacacatcccgatttggcagcaaacctgggttgcagttcattggcatgcatgagaaaggcattatattcaacaataa

SEQ ID NO: 37 - DNA, 84 bp, Sus scrofa
cagtcactacacatcccgattcggcagcaaacctgggttggagtgcatcggcatgtatgagaagggcatcatatttaataatga

SEQ ID NO: 38 - DNA, 84 bp, White lipped peccary
cagtcactacacatcccgattcggcagcaaacctgggttgcagttcattggaatgcatgagaaaggcatcatatttaacaacaa

(19) United States
(12) Patent Application Publication
(10) Pub. No.: US 2005/0038609 A1
(43) Pub. Date: Feb. 17, 2005

(54) Evolution-based functional genomics

(76) Inventor: Steven Albert Benner, Gainesville, FL (US)

Correspondence Address: Steven A. Benner, 1501 NW 68th Terrace, Gainesville, FL, 32605-4147, US

(21) Appl. No.: 10/765,120

(22) Filed: Jan. 28, 2004

Related U.S. Application Data
(63) Continuation-in-part of application No. 07/857,224, filed on Mar. 25, 1992, now Pat. No. 5,958,784.
(63) Continuation-in-part of application No. 08/914,375, filed on Aug. 19, 1997, now Pat. No. 6,377,893.
(63) Continuation-in-part of application No. 09/640,709, filed on Aug. 18, 2000.

(51) Int. Cl. G06F 19/00; G01N 33/48; G01N 33/50

(52) U.S. Cl. 702/19; 703/11

(57) ABSTRACT

The invention concerns methods for applying evolutionary analyses to a set of aligned homologous protein sequences for the purpose of predicting a consensus model for the folded secondary structure of a protein family, identifying distant homologs and denying distant homology, assigning functional behavior to protein families, identifying protein pairs that interact as they function, identifying episodes of sequence evolution where functional behavior within a family is changing, and identifying specific chemical units of the protein that change in concert with changes in functional behavior. Accordingly, this invention is relevant to the use of genomic information to understand homology, fold, behavior and function in proteins.

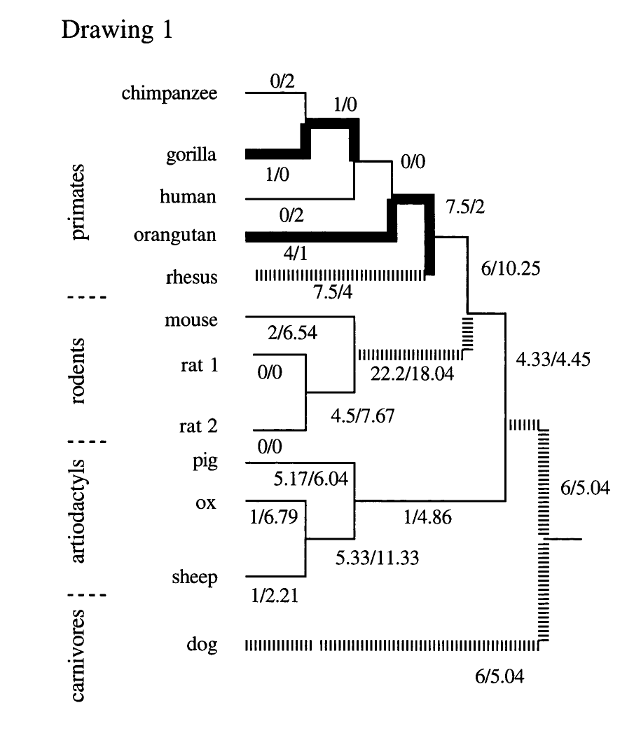

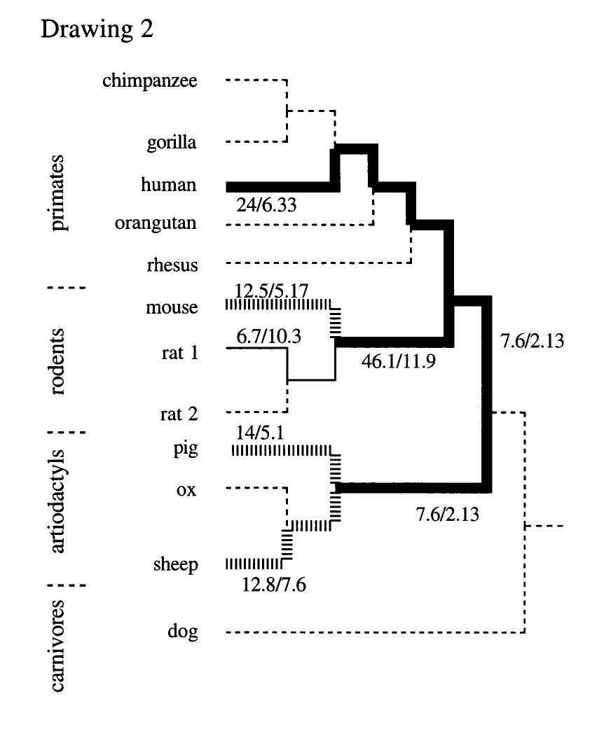

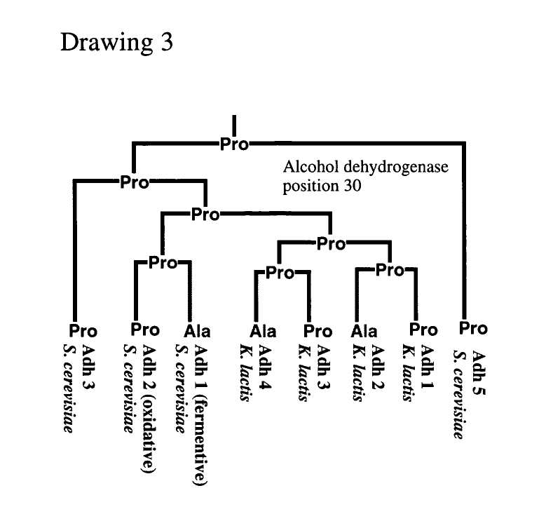

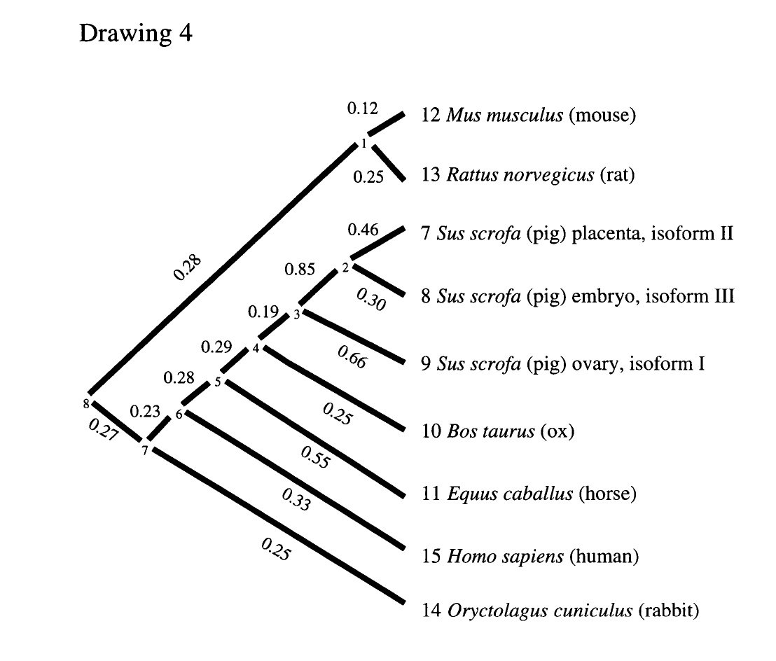

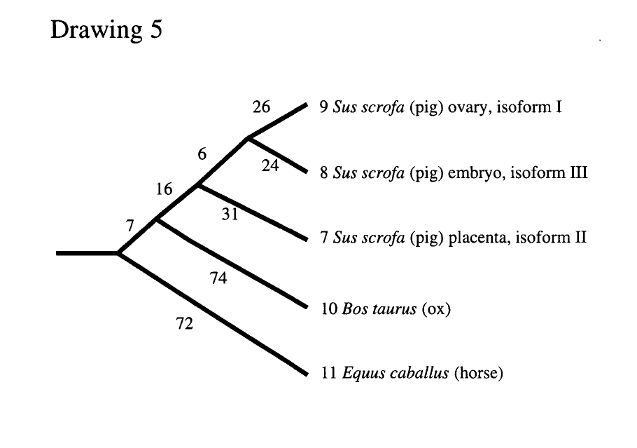

### CROSS REFERENCE TO RELATED APPLICATIONS

This application is a continuation-in-part of application Ser. No. 07/857,224, filed Mar. 25, 1992, and issued as U.S. Pat. No. 05,958,784 on Sep. 28, 1999, and application Ser. No. 08/914,375 filed Aug. 8, 1997, and issued as U.S. Pat. No. 6,377,893 on Apr. 23, 2002, and application Ser. No. 09/640,709, currently pending.

### STATEMENT OF RIGHTS TO INVENTIONS MADE UNDER FEDERALLY-SPONSORED RESEARCH

None

### FIELD OF THE INVENTION

This invention relates to the area of bioinformatics, more specifically to methods for analyzing the sequences of evolutionarily related proteins, and most specifically for identifying evolutionary and functional relationships between proteins and the genes that encode them.

### SUMMARY OF THE INVENTION

As discussed in Ser. No. 08/914,375, the parent for the instant application, the physiological function of a biomolecule is ultimately determined by the contribution that the biomolecule makes to the efforts of the host organism to survive, select a mate (in higher organisms), and reproduce. Determining the physiological function of a protein is not trivial, however. Difficulties in establishing physiological function are discussed at length by Benner and Ellington [Ben88]. Still more difficult is identifying which behaviors of a protein as measured in vitro are relevant for physiological function in vivo. Nevertheless, the identification is important. In vitro behaviors that have relevance to physiological function in vivo are those that are interesting to study for biotechnological, biomedical, or other applications. There is at present in the art no general method for determining what in vitro behaviors are relevant to in vivo function. Processes for determining these behaviors were claimed in the parent application (Ser. No. 08/914,375). A method for making a model for the folded structure of a set of proteins from an evolutionary analysis of a set of aligned homologous protein sequences was claimed in Ser. No. 07/857,224. The instant application concerns methods for using these models. The first method is used to confirm or deny a hypothesis that two proteins are homologous, and is comprised of comparing a predicted structure model for one family of proteins with a predicted structure model for a second family of proteins, or an experimental structure for the second family, and deducing the presence or absence of homology based on the presence or absence of structural similarity flanking key residues in the polypeptide sequence. The second method identifies mutations during the divergent evolution of a protein sequence that are potentially adaptive by identifying episodes during the divergent evolution of a family of proteins where there is a high absolute rate of amino acid substitution, or a high ratio of non-silent substitutions to non-silent substitutions. Amino acids that are changing during this episode are likely to be adaptive. The third is a method for identifying specific in vitro properties of the protein that are likely to play a physiological role in vivo in an organism. This methods involves synthesizing in the laboratory proteins having the reconstructed amino acid sequences of a protein before and after a period of rapid sequence evolution that characterizes adaptive substitution, measuring the in vitro properties of the protein before the episode of rapid sequence evolution, and then measuring the in vivo properties of the protein after the episode of rapid sequence evolution. The in vitro behaviors that remained unchanged through this episode are not likely to have adaptive significance physiologically. The in vitro behaviors that changed through this episode are likely to have adaptive significance physiologically. The fourth concerns method for organizing genome sized sequence databases.

### BRIEF DESCRIPTION OF THE DRAWINGS

Drawing 1. Evolutionary tree showing the evolutionary history of the leptins. Heavy lines show branches with expressed/silent ratios higher than 2. Hatched lines show branches with expressed/silent ratios from 1 to 2. Dotted lines show branches with expressed/silent ratios less than 1, or indeterminate. Numbers on the lines indicate the ratio of expressed/silent changes for that branch. According to the method of the instant invention, a correlation between the episode of high sequence evolution and the evolutionary history of the leptin receptor suggests that the two interact as they function. The multiple alignment, used to derive the tree is shown below. The reconstructed ancestral sequence is from the (now extinct) ancestor of humans, rodents, and ruminants is below the alignment. The sequence as shown here is deterministic; in the work to be performed here, the ancestral sequences are all probabilistic (see text)

| | |
|---|---|
| 080 090 100 110 120 | |
| . \| . \| . \| . \| . \| | |
| RNVIQISNDLENLRDLLHVLAFSKSCHLPWASGLETLDSLGGVLEASGYS | human |
| RNMIQISNDLENLRDLLHVLAFSKSCHLPWASGLETLDSLGGVLEASGYS | chimp |
| RNMIQISNDLENLRDLLHVLAFSKSCHLPWASGLETLDSLGGVLEASGYS | gorilla |
| RNVIQISNDLENLRDLLHVLAFSKSCHLPWASGLETLDRLGGVLEASGYS | orangutan |
| RNVIQISNDLENLRDLLHLLAFSKSCHLPLASGLETLESLGDVLEASLYS | rhesus |
| QNVLQIAHDLENLRDLLHLLAFSKSCSLPQTRGLQKPESLDGVLEASLYS | rat |
| QNVLQIAHDLENLRDLLHLLAFSKSCSLPQTRGLQKPESLDGVLEASLYS | rat |
| QNVLQIANDLENLRDLLHLLAFSKSCSLPQTSGLQKPESLDGVLEASLYS | mouse |
| RNVIQISNDLENLRDLLHLLASSKSCPLPQARGLETLESLGGVLEASLYS | ancestor X |
| RNVIQISNDLENLRDLLHLLASSKSCPLPQARALETLESLGGVLEASLYS | pig |
| RNVIQISNDLENLRDLLHLLAASKSCPLPQVRALESLESLGVVLEASLYS | sheep |
| RNVVQISNDLENLRDLLHLLAASKSCPLPQVRALESLESLGVVLEASLYS | ox |
| RNVVQISNDLENLRDLLHLLASSKSCPLPRARGLETFESLGGVLEASLYS | dog |

Drawing 2. Evolutionary tree showing the evolutionary history of the leptin receptors. Heavy lines show branches with expressed/silent ratios higher than 2. Hatched lines show branches with expressed/silent ratios from 1 to 2. Thin lines show branches with expressed/silent ratios less than 1, or indeterminate. Numbers on the lines indicate the ratio of expressed/silent changes for that branch. Dotted lines indicate branches to the sequence that were not known in 1997, when this analysis was prepared.

Drawing 3. An example of homoplasy taken from the evolution of alcohol dehydrogenase from yeast (position 30). At at least three points in the tree, a P->A substitution occurred independently.

Drawing 4. A sub-tree for the aromatases from 17 vertebrates, exlucing fish, including mammals, built by a Darwin [Gon91] based on an analysis of amino acid sequences. Numbers on the branches are the Kₐ/Kₛ ratios evaluated using the methods of Fitch [Fit71] to reconstruct intermediate evolutionary states and Li et al. [Li85]. The key is given below, together with the multiple sequence alignment used to calculate the tree.

| | |
|---|---|
| 1. | Tilapia nilotica (rainbow trout), GenBank g1613859, mRNA (Chang et al., 1997) |
| 2. | Oryzias latipes (medaka), GenBank g1786171, ovarian follicle mRNA (Tanaka et |
| | al., 1995) |
| 3. | Danio rerio (zebrafish), GenBank g2306966 aromatase mRNA |
| 4. | Carassius auratus (goldfish) ovary, GenBank g2662330, ovarian mRNA |
| 5. | Ictalurus punctatus (channel catfish), GenBank g912802 (Trant, 1994) |
| 6. | Carassius auratus (goldfish) brain, GenBank g2662328, brain mRNA |
| 7. | Sus scrofa (pig) placental, isoform 2, GenBank g1762232, mRNA (Choi et al., |
| | 1997a) |
| 8. | Sus scrofa (pig) embryo, isoform 3, GenBank g1244543, mRNA (Choi et al., 1996) |
| 9. | Sus scrofa (pig) ovary, isoform 1, GenBank g1928957, mRNA (Conley et al., 1997) |
| 10. | Bos taurus (ox), GenBank g665546, mRNA (Hinshelwood et al., 1993) |
| 11. | Equus caballus (horse), GenBank g2921277, mRNA (Boerboom et al. 1997) |
| 12. | Mus musculus (mouse), GenBank g3046857, mRNA (Terashima et al. 1991) |
| 13. | Rattus norvegicus (rat), GenBank g203804, mRNA (Hickey et al., 1990) |
| 14. | Oryctolagus cuniculus (rabbit), GenBank g1240042, mRNA (Delarue et al, 1996) |
| 15. | Homo sapiens (human), GenBank g28846, mRNA (Harada, 1988) |
| 16. | Gallus gallus (chicken), GenBank g211703 (McPhaul et al., 1988) |
| 17. | Poephila guttata (zebra finch), GenBank g926845, ovary mRNA (Shen et al., 1994) |
| | 010 020 030 040 050 060 070 080 |
| | \| \| \| \| \| \| \| \| |
| 1 | MVLEMLNPMHYKVTSMVSEVVPFASIAVLLLTGFLLLVWNYKNTS-SIPGPGYFLGIGPLISYLRFLWMGIGSACNYYNK |
| 2 | MFLEMLNPMQYNVTIMVPETVTVSAMPLLLIMGLLLLIWNCESSS-SIPGPGYCLGIGPLISHGRFLWMGIGSACNYYNK |
| 3 | MILEMLNPMHYNLTSMVPEVMPVATLPILLLTGFLFFVWNHEETS-SIPGPGYCMGIGPLISHLRFLWMGLGSACNYYNK |
| 4 | VLELLMQAHNSSYGAQDNVCGAMATLLLLLLCLLLAIRHHWTEAKDHVPGPCFLLGLGPLLSYCRLIWSGIGTASNYYNS |
| 5 | -MEEVLKGTVNFAATVQVTLMALTGTLLLILLHRIFTAKNWRNQS-GVPGPGWLLGLGPIMSYSRFLWMGIGSACNYYNE |
| 6 | VVDLLIQRAHNGTERAQDNACGATATILLLLLCLLLAIRHHRPHKSHIPGPSFFFGLGPVVSYCRFIWSGIGTASNYYNS |
| 7 | MVLEMLNPMYYKITSMVSEVVPFASIAVLLLTGFLLLLWNYENTS-SIPSPGYFLGIGPLISHFRFLWMGIGSACNYYNE |
| 8 | ----LVSIAPNTTVGLP-SGIPMATRSLILLVCLLLMVWSHSEKK-TIPGPSFCLGLGPLMSYLRFIWTGIGTASNYYNN |
| 9 | MVLEMLNPMN--ISSMVSEAVLFGSIAILLLIGLLLWVWNYEDTS-SIPGPGYFLGIGPLISHFRFLWMGIGSACNYYNK |
| 10 | MVLEMLNPMHFNITTMVPAAMPAATMPILLLTCLLLLIWNYEGTS-SIPGPGYCMGIGPLISYARFLWMGIGSACNYYNK |
| 11 | VMEILLREARNGTDPRYENPRG-ITLLLLLCLVLLLTVWNRHEKKCSIPGPSFCLGLGPLMSYCRFIWMGIGTASNYYNE |
| 12 | MVLETLNPLHYNITSLVPDTMPVATVPILILMCFLFLIWNHEETS-SIPGPGYCMGIGPLISHGRFLWMGVGNACNYYNK |
| 13 | ----VVARSLCDLKCHPIDGISMATRTLILLVCLLLVAWSHTDKK-IVPGPSFCLGLGPLLSYLRFIWTGIGTASNYYNN |
| 14 | MLLEVLNPRHYNVTSMVSEVVPIASIAILLLTGFLLLVWNYEDTS-SIPGPSYFLGIGPLISHCRFLWMGIGSACNYYNK |
| 15 | MVLEMLNPIHYNITSIVPEAMPAATMPVLLLTGLFLLVWNYEGTS-SIPGPGYCMGIGPLISHGRFLWMGIGSACNYYNR |
| 16 | --------------------MPVATVPIIILICFLFLIWNHEETS-SIPGPGYCMGIGPLISHGRFLWMGVGNACNYYNK |
| 17 | MFLEMLNPMHYNVTIMVPETVPVSAMPLLLIMGLLLLIRNCESSS-SIPGPGYCLGIGPLISHGRFLWMGIGSACNYYNK |
| | 090 100 110 120 130 140 150 160 |
| | \| \| \| \| \| \| \| \| |
| 1 | TYGEFIRVWIGGEETLIISKSSSVFHVMKHSHYTSRFGSKPGLQFIGMHEKGIIFNNNPVLWKAVRTYFMKALSGPGLVR |
| 2 | MYGEFMRVWISGEETLIISKSSSMFHVMKHSHYISRFGSKRGLQCIGMHENGIIFNNNPSLWRTIRPFFMKALTGPGLVR |
| 3 | MYGEFVRVWISGEETLVISKSSSTFHIMKHDHYSSRFGSTFGLQYMGMHENGVIFNNNPAVWKALRPFFVKALSGPSLAR |
| 4 | KYGDIVRVWINGEETLILSRSSAVYHVLRKSLYTSRFGSKLGLQCIGMHEQGIIFNSNVALWKKVRTRYAKALTGPGLQR |
| 5 | KYGSIARVWISGEETFILSKSSAVYHVLKSNNYTGRFASKKGLQCIGMFEQGIIFNSNMALWKKVRTYFTKALTGPGLQK |
| 6 | KYGDIVRVWINGEETLILSRSSAVYHVLRKSLYTSRFGSKLGLQCIGMHEQGIIFNSNVALWKKVRAFYAKALTGPGLQR |
| 7 | MYGEFMRVWIGGEETLIISKSSSVFHVMKHSHYTSRFGSKPGLECIGMYEKGIIFNNDPALWKAVRTYFMKALSGPGLVR |
| 8 | KYGDIVRVWINGEETLILSRASAVHHVLKNRKYTSRFGSKQGLSCIGMNEKGIIFNNNVALWKKIRTYFTKALTGPNLQQ |
| 9 | MYGEFMRVWIGGEETLIISKSSSIFHIMKHNHYTCRFGSKLGLECIGMHEKGIMFNNNPALWKAVRPFFTKALSGPGLVR |
| 10 | MYGEFIRVWICGEETLIISKSSSMFHVMKHSHYVSRFGSKPGLQCIGMHENGIIFNNNPALWKVVRPFFMKALTGPGLVQ |
| 11 | KYGDMVRVWISGEETLVLSRPSAVYHVLKHSQYTSRFGSKLGLQCIGMHEQGIIFNSNVTLWRKVRTYFAKALTGPGLQR |
| 12 | TYGDFVRVWISGEETFIISKSSSVSHVMKHWHYVSRFGSKLGLQCIGMYENGIIFNNNPAHWKEIRPFFTKALSGPGLVR |
| 13 | KYGDIVRVWINGEETLILSRSSAVHHVLKNGNYTSRFGSIQGLSYLGMNERGIIFNNNVTLWKKIRTYFAKALTGPNLQQ |
| 14 | MYGEFMRVWVCGEETLIISKSSSMFHVMKHSHYISRFGSKLGLQFIGMHEKGIIFNNNPALWKAVRPFFTKALSGPGLVR |
| 15 | VYGEFMRVWISGEETLIISKSSSMFHIMKHNHYSSRFGSKLGLQCIGMHEKGIIFNNNPELWKTTRPFFMKALSGPGLVR |
| 16 | TYGEFVRVWISGEETFIISKSSSVFHVMKHWNYVSRFGSKLGLQCIGMYENGIIFNNNPAHWKEIRPFFTKALSGPGLVR |
| 17 | MYGEFMRVWISGEETLIISKSSSMVHVMKHSNYISRFGSKRGLQCIGMHENGIIFNNNPSLWRTVRPFFMKALTGPGLIR |
| | 170 180 190 200 210 220 230 240 |
| | \| \| \| \| \| \| \| \| |
| 1 | MVTVCADSITKHLDKLEEVRNDLGYVDVLTLMRRIMLDTSNNLFLGIPLDEKAIVCKIQGYFDAWQALLLKPDIFFKIP- |
| 2 | MVEVCVESIKQHLDRLGEVTDTSGYVDVLTLMRHIMLDTSNMLFLGIPLDESAIVKKIQGYFNAWQALLIKPNIFFKIS- |
| 3 | MVTVCVESVNNHLDRLDEVTNALGHVNVLTLMRRTMLDASNTLFLRIPLDEKNIVLKIQGYFDAWQALLIKPNIFFKIS- |
| 4 | TLEICITSTNTHLDNLSHLMDARGQVDILNLLRCIVVDISNRLFLGVPLNEHDLLQKIHYFDTWQTLVLIKPDVYFRLAW |
| 5 | SVDVCVSATNKQLNVLQEFTDHSGHVDVLNLLRCIVVDVSNRLFLRILPNEKDLLIKIHRYFSTWQAVLIQPDVFFRLN- |
| 6 | TMEICTTSTNSHLDDLSQLTDAQGQLDILNLLRCIVVDVSNRLFLGVPLNEHDLLQKIHKYFDTWQTVLIKPDVYFRLD- |
| 7 | MVTVCADSITKHLDKLEEVRNDLGYVDVLTLMRRIMLDTSNNLFLGIPLDEKAIVCKIQGYFDAWQALLLKPEFFFKFS- |
| 8 | TVEVCVTSTQTHLDNLSSL----SYVDVLGFLRCTVVDISNRLFLGVPVDEKELLQKIHKYFDTWQTVLIKPDIYFKFS- |
| 9 | MVTVCADSITKHLDKLEEVRNDLGYVDVLTLMRRIMLDTSNNLFLGIPMDESAIVVKIQGYFDAWQALLLKPNIFFKIS- |
| 10 | MVAICVGSIGRHLDKLEEVTTRSGCVDVLTLMRRIMLDTSNTLFIGIPMDESAIVVKIQGYFDAWQALLLKPNIFFKIS- |
| 11 | TLEICTMSTNTHLDGLSRLTDAQGHVDVLNLLRCIVVDISNRLFLDVPLNEQNLLFKIHRYFETWQTVLIKPDFYFRLK- |
| 12 | MIAICVESTTEHLDRLQEVTTELGNINALNLMRRIMLDTSNKLFLGVPLDENAIVLKIQNYFDAWQALLLKPDIFFKIS- |
| 13 | TVDVCVSSIQAHLDHLDSL----GHVDVLNLLRCTVLDISNRLFLNVPLNEKELMLKIQKYFHTWQDVLIKPDIYFKFR- |
| 14 | MVTICADSITKHLDRLEEVCNDLGYVDVLTLMRRIMLDTSNMLFLGIPLDESAIVVNIQGYFDAWQALLLKPDIFFKIS- |
| 15 | MVTVCAESLKTHLDRLEEVTNESGYVDVLTLLRRVMLDTSNTLFLRIPLDESAIVVKIQGYFDAWQALLIKPDIFFKIS- |
| 16 | MIAICVESTIVHLDKLEEVTTEVGNVNVLNLMRRIMLDTSNKLFLGVPLDESAIVLKIQNYFDAWQALLLKPDIFFKIS- |
| 17 | MVEVCVESIKQHLDRLGDVTDNSGYVDVVTLMRHIMLDTSNTLFLGIPLDESSIVKKIQGYFNAWQALLIKPNIFFKIS- |
| | 250 260 270 280 290 300 310 320 |
| | \| \| \| \| \| \| \| \| |
| 1 | WLYRKYEKSVKDLKEDMEILIEKKRRRIFTAEKLEDCMDFATELILAEKRGELTKENVNQCILEMLIAAPDTMSVTVFFM |
| 2 | WLYRKYERSVKDLKDEIAVLVEKKRHKVSTAEKLEDCMDFATDLIFAERRGDLTKENVNQCILEMLIAAPDTMSVTLYFM |
| 3 | WLSRKHQKSIKELRDAVGILAEEKRHRIFTAEKLEDHVDFATDLIKAEKRGELTKENVNQCILEMMIAAPDTLSVTVFFM |
| 4 | WLHGKHKRDAQELQDAIAALIEQKRVQLTRAEKFDQ-KDFTGELIFAQSHGELSTENVRQCVLEMIIAAPDTLSISLFFM |
| 5 | FVYKKYHLAAKELQDEMGKLVEQKRQAINNMEKLDE-TDFATELIFAQNHDELSVDDVRQCVLEMVIAAPDTLSISLFFM |
| 6 | WLHRKHKRDAQELQDAITALIEQKKVQLAHAEKLDH-LDFTAELIFAQSHGELSAENVRQCVLEMVIAAPDTLSISLFFM |
| 7 | WLYKKHKESVKDLKENMEILIEKKRCSIITAEKLEDCMDFATELILAEKRGELTKENVNQCILEMLIAAPDTLSVTVFFM |
| 8 | WIHQRHKTAAQELQDAIESLVERKRKEMEQAEKLDN-INFTAELIFAQGHGELSAENVRQCVLEMVIAAPDTLSISLFFM |
| 9 | WLYRKYEKSVKDLKDAMEILIEEKRHRISTAELKEDSMDFTTQLIFAEKRGELTKENVNQCVLEMMIAAPDTMSITVFFM |
| 10 | WLYKKYEKSVKDLKDAIDILVEKKRRRISTAEKLEDHMDFATNLIFAEKRGDLTRENVNQCVLEMLIAAPDTMSVSVFFM |
| 11 | WLHDKHRNAAQELHDAIEDLIEQKRTELQQAEKLDN-LNFTEELIFAQSHGELTAENVRQCVLEMVIAAPDTLSISVFFM |
| 12 | WLCKKYKDAVKDLKGAMEILIEQKRQKLSTVEKLDEHMDFASQLIFAQNRGDLTAENVNQCVLEMMIAAPDTLSVTLFFM |
| 13 | WIHHRHKTATQELQDAIKRLVDQKRKNMEQADKLDN-INFTAELIFAQNHGELSAENVTQCVLEMVIAAPDTLSLSLFFM |
| 14 | WLCRKYEKSVKDLKDAMEILIAEKRHRISTAEKLEDSIDFATELIFAEKRGELTREVNVQCILEMLIAAPDTMSVSVFFM |
| 15 | WLYKKYEKSVKDLKDAIEVLIAEKRRRISTEEKLEECMDFATELILAEKRGDLTRENVNQCILEMLIAAPDTMSVSLFFM |
| 16 | WLCKKYEEAAKDLKGAMEILIEQKRQKLSTVEKLDEHMDFASQLIFAQNRGDLTAENVNQCVLEMMIAAPDTLSVTLFIM |
| 17 | WLYRKYERSVKDLKDEIEILVEKKRQKVSSAEKLEDCMDFATDLIFAERRGDLTKENVNQCILEMLIAAPDTMSVTLYVM |
| | 330 340 350 360 370 380 390 400 |
| | \| \| \| \| \| \| \| \| |
| 1 | LFLIAKHPQVEEELMKEIQTVVGERDIRNDDMQKLEVVENFIYESMRYQPVVDLVMRKALEDDVIDGYPVKKGTNIILNI |
| 2 | LLLVAEYPEVEAAILKEIHTVVGDRDIKIEDIQNLKVVENFINESMRYQPVVDLVMRRALEDDVIDGYPVKKGTNIILNI |
| 3 | LCLIAQHPKVEEALMKEIQTVLGERDLKNDDMQKLKVMENFINESMRYQPVVDIVMRKALEDDVIDGYPVKKGTNIILNI |
| 4 | LLLLKQNPDVELKILQEMNAVLAGRSLQHSHLSGLHILESFINESLRFHPVVDFTMRRALDDDVIEGYEVKKGTNIILNV |
| 5 | LLLLKQNSVVEEQIVQEIQSQIGERDVESADLQKLNVLERFIKESLRFHPVVDFIMRRALEDDEIDGYRVAKTGNLILNI |
| 6 | LLLLKQNPDVELKILQEMDSVLAGQSLGHSHLSKLQILESFINESLRFHPVVDFTMRRALDDDVIEGYNVKKGTNIILNV |
| 7 | LFLIAKHPQVEEAIVKEIQTVIGERDIRNDDMQKLKVVENFIYESMRYQPVVDLVMRKALEDDVIDGYPVKKGTNILLNI |
| 8 | LLLLKQNPHVELQLLQEIDTIVGDSQLQNQDLQKLQVLESFINECLRFHPVVDFTMRRALFDDIIDGHRVQKGTNIILNT |
| 9 | LFLIANHPQVEEELMKEIYTVVGERDIRNDDMQKLKVVENFIYESMRYQPVVDFVMRKALEDDVIDGYPVKKGTNIILNI |
| 10 | LFLIAKHPSVEEAIMEEIQTVVGERDIRIDDIQKLKVVTNFIYESMRYQPVVDLVMRKALEDDVIDGYPVKKGTNIILNI |
| 11 | LLLLKQNAEVERRILTEIHTVLDGTELQHSHLSQLHVLECFINEALRFHPVVDFSYRRALDDDVIEGFRVPRGTNIILNV |
| 12 | LILIAEHPTVEEEMMREIETVVGDRDIQSDDMPNLKIVENFIYESMRYQPVVDLIMRKALQDDVIDGYPVDDGTNIILNI |
| 13 | LLLLKQNPHVEPQLLQEIDAVVGERQLQNQDLHKLQVMESFIYECLSFHPVVDFTMRRALSDDIIEGYRISKGTNIILNT |
| 14 | LFLIAKHPQVEEAIIREIQTVVGERDIRIDDMQKLKVVENFINESMRYQPVVDLVMRKALEDDVIDGYPVKKGTNIILNL |
| 15 | LFLIAKHPNVEEAIIKEIQTVIGERDIKIDDIQKLKVMENFIYESMRYQPVVDLVMRKALEDDVIDGYPVKKGTNIILNI |
| 16 | LILIADDPTVEEKMMREIETVMGDREVQSDDMPNLKIVENFIYESMRYQPVVDLIMRKALQDDVIDGYPVKKGTNIILNI |
| 17 | LLLIAEYPEVETAILKEIHTVVGDRDIRIGDVQNLKVVENFINESLRYQPVVDLVMRRALEDDVIDGYPVKKGTNIILIN |
| | 410 420 430 440 450 460 470 480 |
| | \| \| \| \| \| \| \| \| |
| 1 | GRMHRLEFFPKPNEFTLENFAKNVPYR-YFQPFGFGPRACAGDYIAMVMMKVTLVILLRRFQVQTPQDRCVEKMQKKNDL |
| 2 | GRMHRLEYFPKPNEFTLENFEKNVPYR-YFQPFGFGPRGCAGKYIAMVMMKVVLVTLLRRFQVKTLQKRCIENIPKKNDL |
| 3 | GRMHKLEFFPKPNEFTLENFEKNVPYR-YFQPFGFGPRSCAGKFIAMVMMKVMLVSLLRRFHVKTLQGNCLENMQKTNDL |
| 4 | GRMKRSEFFPKPNEFSLDNFQKNVPSR-FFQPFGSGPRSCVGKHIAMVMMKSILVTLLSRFSVCPVKGCTVDSIPQTNDL |
| 5 | GRMHKSEFFQKPNEFNLENFENTVPSR-YFQPFGCGPRACVGKHIAMVMTKAILVTLLSRFTVCPRHGCTVSTIKQTNNL |
| 6 | GRMHRSEFFSKPNQFSLDNFHKNVPSR-FFQPFGSGPRSCVGKHIAMVMMKSILVALLSRFSVCPMKACTVENIPQTNNL |
| 7 | GRMHRLEFFPKPNEFTLENFAKNVPYR-YFQPFGFGPRACAGKYIAMVMMKVTLVILLRRFQVQTPQDRCVEKMQKKNDL |
| 8 | GRMHRTEFFHKANEFSLENFQKNTPRR-YFQPFGSGPRACVGRHIAMVMMKSILVTLLSQYSVCPHEGLTLDCLPQTNNL |
| 9 | GRMHRLEFFPKPNEFTLENFAKNVPYR-YFQPFGFGPRACAGKYIAMVMMKVILVTLLRRFQVQTQQGQCVEKMQKKNDL |
| 10 | GRMHRLEFFPKPNEFTLENFAKNVPYR-YFQPFGFGPRGCAGKYIAMVMMKVILVTLLRRFQVKALQGRSVENIQKKNDL |
| 11 | GRMHRSEFYPKPADFSLDNFNKPVPSR-FFQPFGSGPRSCVGKHIAMVMMKAVLLMVLSRFSVCPEESCTVENIAHTNDL |
| 12 | GRMHKLEFFPKPNEFSLENFEKNVPSR-YFQPFGFGPRSCVGKFIAMVMMKAILVTLLRRCRVQTMKGRGLNNIQKNNDL |
| 13 | GRMHRTEFFLKGNQFNLEHFENNVPRPPTFQPFGSGPRACIGKHMAMVMMKSILVTLLSQYSVCTHEGPILDCLPQTNNL |
| 14 | GRMHRLEFFPKPNEFTLENFAKNVPYR-YFQPFGFGPRGCAGKYIAMVMMKVVLVTLLRRFHVQTLQGRCVEKMQKKNDL |
| 15 | GRMHRLEFFPKPNEFTLENFAKNVPYR-YFQPFGFGPRGCAGKYIAMVMMKAILVTLLRRFHVKTLQGQCVESIGKIHDL |
| 16 | GRMHKLEFFPKPNEFSLENFEKNVPSR-YFQPFGFGPRGCVGKFIAMVMMKAILVTLLRRCRVQTMKGRGLNNIQKNNDL |
| 17 | GRMHRLEYFPKPNEFTLENFEKNVPRY-YFQPFGFGPRSCAGKYIAMVMMKVVLVTLLKRFHVKTLQKRCIENMPKNNDL |
| | 490 |
| | \| |
| 1 | SLHPDETSG |
| 2 | SLHPNEDRH |
| 3 | ALHPDESRS |
| 4 | SQQPVEEPS |
| 5 | SMQPVEEDP |
| 6 | SQQPVEEPS |
| 7 | SLHPDETSG |
| 8 | SQQPVEHHQ |
| 9 | SLHPHETSG |
| 10 | SLHPDETSD |
| 11 | SQQPVEDKH |
| 12 | SMHPIERQP |
| 13 | SQQPVEHQQ |
| 14 | SLHPDETRD |
| 15 | SLHPDETKN |
| 16 | SMHPIERQP |
| 17 | SLHLDEDSP |

Drawing 5. An evolutionary tree built from neutral evolutionary distances (NEDs) calculated by assuming a first order approach to equilibrium for codon usage at two fold redundant silent sites. Numbers on branches of the tree correspond to evolutionary time (in million years) estimated from the NEDs using a first order rate constant for pyrimidine-pyrimidine transitions of 3×10⁻⁹ changes per base per year.

### DETAILED DESCRIPTION OF THE INVENTION

This disclosure describes the classes of tools that permit the scientist to generate experimentally testable hypotheses concerning the function of a protein starting from an evolutionary analysis. These are outlined below:
I. Tools that detect change in function within a family of proteins.
   A. Ratios of silent to non-silent substitution along specific branches of an evolutionary tree including tools that address normalization issues.
   B. Covarion behavior, in which individual residues display different mutability in different branches of a tree.
   C. Detecting high absolute rates of amino acid substitution, changes per unit time.
II. Tools that detect conservation of function within a family of proteins.
   A. Compensatory changes
   B. Homoplasy
   C. Absolute conservation within a defined evolutionary distance
III. Tools that identify individual residues involved in changes in functionally significant behavior.
   A. Residues changing in episodes with high Kₐ/Kₛ values, minus residues changing in episodes with low Kₐ/Kₛ values
   B. Residues displaying covarion behavior
   C. Mapping these residues on to models for the secondary, tertiary, and quaternary structure of proteins.
IV. Tools that identify individual residues involved in conserved of functionally significant behavior
   A. Residues suffering compensatory changes
   B. Residues displaying homoplasy
   C. Mapping these residues on to models for the secondary, tertiary, and quaternary structure of proteins.
V. Tools that involve correlation between the evolutionary histories of two families of proteins
   A. Correlating the topology of evolutionary trees in two families of proteins
   B. Correlating the connectivity of proteins in a gene family
   C. Dating events in the molecular history
   D. Correlating evolutionary events in two protein families occuring at approximately the same time
   E. Correlating evolutionary events in two protein families that are associated with analogous behavior involving expressed/silent ratios
VI. Tools that involve correlation between the evolutionary history of a family of proteins and the evolutionary history of the organism as known from some source other than genomic sequence data, including paleontology, geology, ecology, ontogeny, phylogeny, or systematics (collectively known as the “non-genomic record”.
   A. Correlating the topology of an evolutionary trees and the non-genomic record.
   B. Correlating features of patterns of evolution in specific branches in the evolutionary tree with the non-genomic record
   C. Correlating evolutionary events in several protein families occuring at approximately the same time with the non-genomic record

Many of these tools are new in this disclosure. Others were disclosed in Ser. No. 07/857,224 and Ser. No. 08/914,375 and are claimed here for the first time. In many cases, elements of novelty and utility can be found by combining these tools. This disclosure will systematically indicate the Applicant's presently preferred combinations, with statements of where the Applicant believes that the state of the prior art requires reference to the priority dates of parent applications, where it does not.

All of the tools have in common the same starting point, a basic evolutionary model based on three parts:
(a) An evolutionary tree that shows the familial relationship between the members of the protein family,
(b) A multiple alignment of the sequences of members of the protein family, which shows the evolutionary relationship between the individual amino acids in the sequences, and
(c) The sequences of ancient proteins that were the ancestors of the contemporary proteins in the family.

Each element of an evolutionary model requires the other two in the reconstruction process. Accordingly, processes for constructing an evolutionary model for a protein family are frequently iterative. These processes are well know in the art, and include parsimony tools [Fit67], maximum likelihood tools [Gon91][Gon96][Tho92], tools for evaluating the probability of an evolutionary model [Gon96], and gamma models [Swo96] [Li97].

Ser. No. 08/914,375 disclosed the step-by-step procedure in which the basic evolutionary model for a family of proteins is constructed to support the tools outlined above.

(a) A multiple alignment, an evolutionary tree, and ancestral sequences at nodes in the tree are constructed by methods well known in the art for a set of homologous proteins. These three elements of the description are interlocking, as is well known in the art. The presently preferred methods of constructing ancestral sequences for a given tree is the maximum parsimony methods, as implemented (for example) in the commercially available program MacClade [Mad92]. Alternative methods for reconstructing evolutionary intermediates can now be found with the PAUP program [Swo96][and using the maximum likelihood method of the PAML program [Yan97]. Trees are compared based on their scores using either maximum parsimony or maximum likelihood criteria, and selected based on considerations of score and correspondence to known facts. Step (a) is part of the process used to generate the predictions of secondary structure using the method disclosed in Ser. No. 07/857,224.

(b) A corresponding multiple alignment is constructed by methods well known in the art for the DNA sequences that encode the proteins in the protein family. The multiple alignment is constructed in parallel with the protein alignment. In regions of gaps or ambiguities, the amino acid sequence alignment can be adjusted to give the alignment with the most parsimonious DNA tree. The presently preferred method of constructing ancestral DNA sequences for a given tree is the maximum parsimony method. The DNA and protein trees and multiple alignments must be congruent, meaning that when amino acids are aligned in the protein alignment, the corresponding codons are aligned in the DNA alignment. Likewise, the connectivity of the two evolutionary trees must show the same evolutionary relationships. In regions where the connectivity of the amino acid tree is not uniquely defined by the amino acid sequences, the tree that gives the most parsimonious DNA tree is used to decide between two trees or reconstructions of equal value. Finally, the ancestral amino acids reconstructed at nodes in the tree must correspond to the reconstructed codons at those nodes. When the ancestral sequences are ambiguous, and where the DNA sequences cannot resolve the ambiguity, the reconstructed DNA sequences must be ambiguous in parallel. Approximate reconstructions are valuable even when exact reconstructions are not possible from available data, and the tree is preferably constrained to correspond to evolutionary relationships between proteins inferred from biological data (e.g., cladistics).

(c) Mutations in the DNA sequences are then assigned to each branch of the DNA evolutionary tree. These may be fractional mutations to reflect ambiguities in the sequences at the nodes of the tree. When ambiguities are encountered, alternatives are weighted equally. Mutations along each branch are then assigned as being “silent”, meaning that they do not have an impact on the encoded protein sequence, and “expressed”, meaning that they do have an impact on the encoded protein sequence. Fractional assignments are made in the case of ambiguities in the reconstructed sequences at nodes in a tree.

As disclosed in Ser. No. 08/914,375, the quality of a multiple alignment and the precision of the reconstructed ancestral sequences decreases if proteins are included in the family with sequences diverging by over 150 PAM units, where a PAM unit is the number of point accepted mutations per 100 amino acids. For this reason, families are most preferably constructed with a tree “width” (the distance between the two most divergent proteins in the family) of 150 PAM units or less. Some variation is, of course, desired. Therefore, the PAM width of the tree is preferably more than 50 PAM units. Also referred are well articulated trees. In principle, the more sequences in the tree, the more valuable an evolutionary analysis of the tree becomes.

With the emergence of massive amounts of sequence information as a result of genome projects, the ability to construct detailed evolutionary histories of protein families will increase. This will make the inventions disclosed herein of still greater value, as is appreciated by one of ordinary skill in the art.

One key inventive feature of Ser. No. 07/857,224 was that an evolutionary analysis had additional value when placed within well defined. One key inventive feature of Ser. No. 08/914,375 was that an evolutionary analysis gained additional value when it involved analysis of explicitly reconstructed intermediates in the evolutionary tree. These inventive concepts are at the core of all of the tools outlined above.

Another key inventive feature of Ser. No. 08/914,375 was that an evolutionary analysis gained additional value when it is correlated with the non-genomic record. This inventive concepts is at the core of all of the tools in class VI outlined above.

Another key inventive feature of Ser. No. 08/914,375 involved the use of a natural organization to generate a rapidly searchable database. As disclosed in the specification to Ser. No. 08/914,375, when all of the genomes of all of the organisms on planet Earth are completed, all protein sequences will be easily recognizable as members of one of ca. 10,000-100,000 nuclear families, protein sequence modules 50-500 amino acids long that are related by common ancestry. This conclusion reflects the well known fact that all organisms on the planet are descendants of a single ancestor. In the course of producing the diversity of organisms now on Earth, divergent evolution also produced the diversity of molecular genetic sequences within nuclear families.

As disclosed in the specification to Ser. No. 08/914,375, this permits a naturally organized database. The ancestral sequences and the predicted secondary structures associated with the families are surrogates for the sequences and structures of the individual proteins that are members of the family. The reconstructed ancestral sequence represents in a single sequence all of the sequences of the descendent proteins. The predicted secondary structure associated with the ancestral sequence represents in a single structural model all of the core secondary structural elements of the descendent proteins. Thus, the ancestral sequences can replace the descendent sequences, and the corresponding core secondary structural models can replace the secondary structures of the descendent proteins.

This makes it possible to define two surrogate databases, one for the sequences, the other for secondary structures. The first surrogate database is the database that collects from each of the families of proteins in the databases a single ancestral sequence, at the point in the tree that most accurately approximates the root of the tree. If the root cannot be determined, the ancestral sequence chosen for the surrogate sequence database is near the center of mass of the tree. The second surrogate database is a database of the corresponding secondary structural elements. The surrogate databases are much smaller than the complete databases that contain the actual sequences or actual structures for each protein in the family, as each ancestral sequence represents many descendent proteins. Further, because there is a limited number of protein families on the planet, there is a limit to the size of the surrogate databases. Based on our work with partial sequence databases [Gon92], and given more recent data emerging from sequences, we expect there to be on the order of 100,000 families as defined by steps (a) through (e).

Searching the surrogate databases of the instant invention for homologs of a probe sequence thus proceeds in two steps. In the first, the probe sequence (or structure) is matched against the database of surrogate sequences (or structures). As there will be on the order of 100000 families of proteins as defined by steps (a) through (e) after all the genomes are sequenced for all of the organisms on earth, there will be only on the order of 100000 surrogate sequences to search. Thus, this search will be far more rapid than with the complete databases. A probe protein sequence (or DNA sequence in translated form) can be exhaustively matched [Gon92] against this surrogate database (that is, every subsequence of the probe sequence will be matched against every subsequence in the ancestral proteins) more rapidly than it could be matched against the complete database.

Should the search yield a significant match, the probe sequence is identified as a member of one of the families already defined. The probe sequence is then matched with the members of this family to determine where it fits within the evolutionary tree defined by the family. The multiple alignment, evolutionary tree, predicted secondary structure and reconstructed ancestral sequences may be different once the new probe sequence is incorporated into the family. If so, the different multiple alignment, evolutionary tree, and predicted secondary structure are recorded, and the modified reconstructed ancestral sequence and structure are incorporated into their respective surrogate databases for future use.

The advantage of this data structure over those presently used is apparent. As presently organized, sequence and structure databases treat each entry as a distinct sequence. Each new sequence that is determined increases the size of the database that must be searched. The database will grow roughly linearly with the number of organismal genomes whose sequences are completed, and become increasingly more expensive to search.

The surrogate database will not grow linearly. Most of the sequence families are already represented in the existing database. Addition of more sequences will therefore, in most cases, simply refine the ancestral sequences and associated structures. In any case, the total number of sequences and structures in their respective databases will not grow past ca. 100000, the estimate for the total number of sequence families that will be identifiable after the genomes of all organisms on earth are sequenced. If a dramatically new class of organism is identified, this estimate may grow, but not exponentially (as is the growth of the present database).

Since Ser. No. 08/914,375 was filed, other databases have emerged that offer some precomputed families. Most noteworthy are Pfam [Bat00] and ProDom [Cor00].

Ser. No. 07/857,224 disclosed methods to identify residues, secondary structural elements, and evolutionary episodes that are involved in functional adaptation

Further, during episodes of rapid sequence evolution, amino acid substitutions will be concentrated in secondary structural elements defined by the method claimed in Ser. No. 07/857,224. These are secondary structural elements that are important in the acquisition of new function. A general method for identifying secondary structural elements that contribute to the origin of new biological function is comprised of identifying an element in the predicted secondary structure model where the corresponding section of the gene has a high ratio of expressed to silent changes.

4. Identification of In Vitro Behaviors that Contribute to Physiological Function.

In vitro experiments in biological chemistry extract data on proteins and nucleic acids (for example) that are removed from their native environment, often in pure or purified states. While isolation and purification of molecules and molecular aggregates from biological systems is an essential part of contemporary biological research, the fact that the data are obtained in a non-native environment raises questions concerning their physiological relevance. Properties of biological systems determined in vitro need not correspond to those in vivo, and properties determined in vitro need have no biological relevance in vivo.

To date, there has been no simple way to say whether or not biological behaviors are important physiologically to a host organism. Even in those cases where a relatively strong case can be made for physiological relevance (for example, for enzymes that catalyze steps in primary metabolism), it has proven to be difficult to decide whether individual properties of that enzymes (k_{cat}, Kₘ, kinetic order, stereospecificity, etc.) have physiological relevance. Especially difficult, however, is to ascertain which behaviors measures in vitro play roles in “higher” function in metazoa, including development, regulation, reproduction, digestion.

A general method to determine whether a behavior measured in vitro is important to the evolution of new physiological function is comprised of the following steps:
(a) Prepare in the laboratory proteins that have the reconstructed sequences corresponding to the ancestral proteins before, during, and after the evolution of new biological function, as revealed by an episode of high expressed to silent ratio of substitution in a protein. This high ratio compels the conclusion that the protein itself serves a physiological role.

(b) Measure in the laboratory the behavior in question in ancestral proteins before, during, and after the evolution of new biological function, as revealed by an episode of high expressed to silent ratio of substitution. Those behaviors that increase during this episode are deduced to be important for physiological function. Those that do not are not.

We now discuss using the basic evolutionary model in the context of tools that generate hypotheses concerning function within and between protein families.

I. Tools that Detect Change in Function within a Family of Proteins.

A. Ratios of Silent to Non-Silent Substitution Along Specific Branches of an Evolutionary Tree Including Tools that Address Normalization Issues.

As discussed in Ser. No. 07/857,224, during the divergent evolution of two proteins from a common ancestor, mutations of two types accumulate. The first have no impact on the ability of the host organism to survive, select a mate, and reproduce; these are called “neutral” mutations. The second influence the behavior of the protein in a way that influences the ability of the organism to survive, select a mate, and reproduce. These are termed “adaptive mutations.” When evolving a new function, proteins undergo an episode of rapid sequence evolution that corresponds to adaptive “positive selection”, as is well known in the art [Kre95].

Given a basic evolutionary model for a protein family, we can begin to search for sequence details that are indicative of function. For example, the genetic code is degenerate. Some mutations randomly introduced into a genome do not alter the encoded amino acid (“silent mutations”). Others do (“non-silent mutations”). When the gene is under no selective pressure at all, it makes no difference to natural selection whether the mutation changes an amino acid or not. Thus, mutations at the level of the gene are (essentially) neutral, and are fixed in a population without regard to whether they are silent or non-silent. The ratio of non-silent to silent changes can be normalized for the number of silent sites in a particular sequence to give Kₐ and Kₛ values.

When the function of a protein is constant, non-silent changes are usually detrimental. Non-silent changes are therefore removed by natural selection. Silent changes are not. The Kₐ/Kₛ value is therefore lower than unity in a protein divergently evolving under a constant set of functional constraints. Indeed, for many proteins with function that has been established early in natural history (such as cytochromes), the ratio approaches zero. At the start of the evolutionary period where the calculation is done, the protein is already doing its job nearly optimally, and neither needs nor wants to change its amino acids. Conversely, if one reconstructs the evolutionary history of a protein, and identifies an episode in that evolution where the non-silent/silent ratio is very much less than one, the genomic analysis suggests that the protein has a conserved function during that episode.

One of ordinary skill in the art will note that this method assumes that codon selection is not strongly selected in metazoa. This is not true in eubacteria, or in highly expressed genes in yeast, for example. However, there is little evidence in metazoa to suggest that codon usage is strongly selected in multicellular plants and animals (metazoa), including mammals, where most of the ORFs needing analysis for a developmental biology program are studied. Therefore, the presently preferred scope for methods involving the analysis of silent substitutions is in multicellular organisms.

The exact opposite is the case when new function (implying, of course, new behaviors as well) is being engineered into a protein during an episode of evolution. Non-silent changes, those where amino acids are replaced at the level of the protein, are the only way to change the behavior of a protein to perform its new role. Natural selection desires non-silent changes, as these create new behaviors. The Kₐ/Kₛ value is high.

The ratio of non-silent to silent changes, normalized for the number of non-silent and silent sites (the Kₐ/Kₛ value) was introduced in the 1980s as a way of detecting change in function between proteins at the leaves of trees[Li97]. It was applied to a large number of cases (for an example, see [McD91][Jol89]). Both the Applicant [Tra96] and Stewart and her coworkers [Mes97] extended this method to analyze reconstructed evolutionary events, calculating Kₐ/Kₛ values between ancestral nodes in an evolutionary tree, and applied it to individual cases (ribonuclease and lysozyme, respectively). Using this approach, if one reconstructs the evolutionary history of a protein, and identifies an episode in that evolution where the Kₐ/Kₛ value is greater than unity, the protein is evolving a new function during that episode.

In practice, Kₐ/Kₛ values are not so easily interpretable. Even when the function of a protein is changing, some residues (such as those holding together the fold) cannot change without destroying the ability of the protein to serve as a scaffold for function. Thus, the Kₐ/Kₛ value for specific sites can be very high during an episode of divergent evolution, perhaps even much higher than unity. But because Kₐ/Kₛ values are calculated for the sequence as a whole, the sites undergoing rapid substitution are counted with “core” sites undergoing slow substitution, giving a Kₐ/Kₛ value for the protein as a whole of less than unity.

Likewise, Kₐ/Kₛ values are assigned to individual branches of an evolutionary tree. If the evolutionary tree is poorly articulated, a single branch may contain both adaptive and conservative episodes of evolution. In this case, the high Kₐ/Kₛ value for the adaptive episode may be diluted by a low Kₐ/Kₛ value for the conservative episode. The second problem will, of course, subside as more and more genome sequence projects are completed.

One solution to this problem involves normalization of the Kₐ/Kₛ values for a protein family. Here, the average Kₐ/Kₛ value for the average branch of the tree is calculated. Thos branches that have a Kₐ/Kₛ value an arbitrary factor higher (the presently preferred factor is two fold higher) are then hypothesized to be undergoing a change in function. More preferably, a statistical analysis is performed where the number of sites undergoing changes is determined for each branch length, the average Kₐ/Kₛ value is calculated, a statistical model is constructed to assess the distribution of Kₐ/Kₛ values on different branches of the tree, and branches that have Kₐ/Kₛ values lying more than two standard deviations above the mean are hypothesized to contain a change in function

Ser. No. 08/914,375 discussed in greater detail the tools based on the fact that the genetic code is degenerate. More than one triplet codon encodes the same amino acid. Therefore, a mutation in a gene can be either silent (not changing the encoded amino acid) or expressed (changing the encoded amino acid). Especially in multicellular organisms, and most particularly in multicellular animals (metazoa), silent changes are not under selective pressure. In contrast, expressed changes at the DNA level, by changing the structure of the protein that the gene encodes, change the property of the protein.

When examining a protein from higher organisms during a period of evolutionary history where, at the outset of the period, the behavior of a protein is optimized for a specific biological function, and where that function remains constant for the protein throughout the period being examined, changes in the DNA sequence that lead to a change in the sequence of the encoded protein (expressed changes) will diminish the survival value of the protein [Ben88] and therefore will be removed by natural selection. During the same period, silent changes will not be removed by natural selection, but will accumulate at an approximately clock-like rate, as silent changes are approximately neutral, especially in higher organisms. Thus, the ratio of expressed to silent changes will be low during a period of evolution of a protein family where the ancestor and its descendants share a common function.

In contrast, in genes for proteins that are neutrally drifting without functional constraints, the expressed/silent ratio will reflect random introduction of point mutations. Given the genetic code and a typical distribution of amino acid codons within the gene, a ratio of expressed to silent changes will be approximately 2.5 during the period of evolution of a protein family where the ancestor and its descendants have no function.

A third situation concerns a period of evolution where a protein is acquiring a new derived function. The amino acid sequence of the protein at the beginning of this episode will be optimized for the ancestral function, rather than the derived function. Thus, changes in the gene that are expressed in changes in the sequence of the encoded protein that improve the behavior of the protein as is required for the new biological function will be selected for. In proteins in such an evolutionary episode seeking new function, natural selection seeks expressed changes, and the ratio of expressed to silent substitutions at the DNA level will be high during the period of evolution of a protein family where the function of the ancestor has changed with a new function emerging in its descendants. Ratios as high as 4:1 or more are known.

In a family of proteins defined by steps (a) through (e) above, individual periods of evolution are defined by lines between nodes on an evolutionary tree. In step (c), silent and expressed point mutations are assigned to individual periods of evolution. Periods of evolution with high ratios of expressed to silent mutations are episodes where physiological function is rapidly changing. Periods of evolution with low ratios of expressed to silent mutations are episodes where physiological function is slowly changing.

Ser. No. 08/914,375 showed the application of this approach applied to the leptin family of proteins. Leptins are present in mice, where they are believed to modulate feeding behavior. Leptin homologs are also present in humans, and the pharmaceutical industry has been excited about exploiting them in the treatment of obesity. The conclusion drawn from this hypothesis is that the leptin protein in humans does not have the same function as the leptin protein in mice.

B. Covarion Behavior, in which Individual Residues Display Different Mutability in Different Branches of a Tree.

Functional changes leave signatures in the patterns of sequence evolution in a protein family. Covarion behavior was detected in alcohol dehydrogenase [Ben89] and superoxide dismutase [Miy95]. In the alcohol dehydrogenase example, sites in the substrate binding pocket were found to have undergone more replacements in the subfamily of enzymes from mammalian livers than in the subfamily of enzymes from yeast. This could be used as evidence for the statement that the function of these dehydrogenases in liver is different from the function in yeast, and correlation with the crystal structure shows that the substrate binding specificity in liver is changing, while the substrate binding specificity for the enzymes in yeast has not.

Covarion behavior indicates changing function. It is therefore expected to correlate positively with events with high Kₐ/Kₛ ratios. Because Kₐ/Kₛ ratios use a silent substitution clock that ticks rapidly, while covarion analysis does not, the two are somewhat complementary.

C. Detecting High Absolute Rates of Amino Acid Substitution, Changes Per Unit Time.

An alternative way to detect changes in function is to measure the number of amino acids substitutions that occur per unit time. This requires that dates be assigned to nodes in an evolutionary tree. This can be done by correlation with the paleontological record, as is well known in the art.

II. Tools that Detect Conservation of Function within a Family of Proteins.

A. Compensatory Changes

The conservation of the overall fold after extensive divergences raises the possibility that amino acid substitutions at one position in a polypeptide chain might be compensated by substitutions elsewhere in a protein. For example, if a Gly at one position inside the folded protein core is replaced by a Trp, it might be necessary to substitute a Trp by a Gly at a position distant in the sequence but near in space to conserve the overall volume of the core, and therefore the overall folded structure. These assume that if a substitution is not compensated, the organism hosting the protein is less fit.

Individual examples of compensatory changes in proteins have been proposed [Oos86], both by analysis of families of natural proteins with known structures [Les80][Les82][Cho82][Alt87a][Alt87b][Bor90] and in proteins into which point mutations have been introduced by site-directed mutagenesis [Lim89][Lim92][Bal93]. In these examples, amino acid residues distant in the sequence but near in three dimensional space in the folded structure have been observed to undergo simultaneous compensatory variation to conserve overall volume, charge, or hydrophobicity.

Compensatory covariation has been used in the prediction of the tertiary folds. For protein kinase [Ben91], for example, an antiparallel beta sheet was predicted for the core of the first domain because of two specific compensatory changes identified in consecutive strands in the predicted secondary structural model. The subsequently determined crystal structure [Kni91] showed not only that antiparallel beta sheet existed, but that the side chains of the two residues undergoing compensatory covariation were indeed in contact.

Systematic studies have suggested, however, that the compensatory covariation generates only a small signal. The early work by Lesk and Chothia with the globin family found that replacements of hydrophobic residues in the core of the protein fold are usually accommodated by small shifts of secondary structural elements rather than by size complementary amino acid substitutions [Les80][Les82][Cho82]. More recent studies have suggested that a weak compensatory covariation signal might exist [Tay94][Shi94][Gob94][Neh94]. Some authors have doubted, however, that the signal is adequate to be useful in structure prediction [Tay94]. Others have been more optimistic [Neh94][Shi94]. More recently, Chelvanayagam et al. pointed out that the signal might be improved if examples of compensatory covariation were sought within explicit evolutionary context [Che97][Che98].

In the literature, compensatory changes have been sought by comparing the sequences of two extant proteins from contemporary organisms. In principle, any position where an amino acid residue had undergone substitution at any point in the time separating the two proteins via the common ancestor might be paired with any other position that had also suffered substitution in this time. Such an approach is problematic because the evolutionary time separating two contemporary protein sequences can be long; in years, it is twice the time since the most recent common ancestor of the two proteins.

A different way to detect compensatory covariation begins with the recognition that a model for the historical past in a protein family can be inferred from a set of homologous protein sequences These models have three parts: (a) an evolutionary tree, which shows the genealogical relationships between individual proteins in the family, (b) a multiple sequence alignment, which shows the evolutionary relationship between individual nucleotides in the genes encoding each family, and (c) reconstructed sequences of ancestral proteins that are evolutionary intermediates in the tree. Through the reconstruction of ancestral sequences, specific changes in a protein sequence can be assigned to (and isolated to) specific branches of the evolutionary tree. Within the context of a reconstructed model for the historical past, compensatory covariation should appear as two substitutions occurring on the same branch of the evolutionary tree. As these branches can be rather short in length, an analysis based on a reconstructed history of a protein family can identify changes that occur nearly simultaneously. These are expected to be true indicators of compensation. In principle, a weak compensatory covariation signal observed by the comparison of extant sequences should be strengthened by examining individual episodes in divergent evolution as reflected by specific branches in the evolutionary tree.

In preliminary studies, we examined 71 families of proteins from the Master Catalog to learn whether reconstructed ancestral sequences will generate a more useful signal for compensatory covariation than can be obtained by examining extant sequences. We noticed anecdotally that covariation was more likely to occur along branches with low Kₐ/Kₛ values. This makes sense, as compensation is necessary only if function is conserved. Case studies developed under this project will test this.

B. Homoplasy

One feature commonly observed in the divergent evolution but not modelled well by even advanced stochastic models is molecular homoplasy, defined as a character similarity that arose independently in different subfamilies of an evolutionary tree [Str00].

Molecular homoplasy is best illustrated by an example (Drawing 3). Homoplasy so defined is the observed phenomenon; no statement is made as to the mechanism by which homoplasy arises. It may reflect selection pressures. The Master Catalog gives us the opportunity to systematically search for molecular homoplasy in the database as a whole.

At one level, homoplasy is simply the statement that selective pressures are forcing the protein to select from a subset of the 20 standard amino acids. Thus, it is similar to the bias that is seen in membrane proteins, for example (where residues are chosen more frequently from a subset of hydrophobic amino acids than in the database as a whole). Homoplasy is more. Not only (in the example) is position 30 limited to A and P, but the selection pressures have toggled between the two more than once in the module's evolutionary history.

This is, of course, a signature that a functional constraint is conserved in the distant branches of the tree protein. For this reason, molecular homoplasy is expected to be a contrarian signature to high Kₐ/Kₛ or non-stationary covarion behavior in a protein. We expect it to occur more frequently with proteins that are not undergoing functional recruitment.

Some informative features are already evident from preliminary work. For example, a preliminary search of 38 protein families with high resolution crystal structures identified over 2000 examples of molecular homoplasy. These were characterized first by the nature of the amino acids identified. A number of very obvious patterns emerged. First, the majority of the examples involve the interchange of hydrophobic side chains of nearly identical volume. The homoplasy involving I and V was the most frequent. It occurred 230 times in the dataset. The I/V molecular homoplasy was far more abundant than the next most popular hydrophobic/hydrophobic homoplasy, F/Y, which was found 68 times, and the I/L hydrophobic/hydrophobic homoplasy, which was found 44 times. As might be expected, the majority of these were buried in the three dimensional structure of the protein.

In the next phase of work we will ask whether these homoplasies are correlated with homoplasies at other positions in the same sequence in the same branches of the trees. If the functional constraint at the amino acid position are sufficient to permit a protein to confer fitness only if it places one of two residues there, then this constraint might be sufficient to cause compensation, also possibly homoplastic, at a second position nearby in the folded structure of the protein. Further, it is necessary to characterize the branch length (NED or PAM) where the changes occur.

The most interesting homoplasies are those that involve multiple steps. For example, the Pro/Gly homoplasy (at the codon level, CCN to GGN) requires two substitutions. Either of these alone creates a change in the encoded amino acid (CGN, Arg, or GCN, Ala). Observing examples of these without observing the intermediates anywhere else in the tree suggests that selection pressure is remarkably strong at this position, even though two amino acids appear to be nearly equally suited to perform function.

Molecular homoplasy indicates a constraint on structure that implies a constant behavior, which in turn implies a constant function. If this is true, it should correlate negatively with Kₐ/Kₛ ratios. That is, homoplasy should be found less frequently in branches separated by a branch with a high Kₐ/Kₛ ratio than in branches not separated by such a branch. Case studies developed under this project will develop ways to exploit such a correlation.

C. Absolute Conservation within a Defined Evolutionary Distance

As disclosed in Ser. No. 07/857,224, residues that are conserved over an entire evolutionary tree are presumed (at the level of hypothesis) to be important for function, especially if they are chosen from the group consisting of Asp, Lys, Arg, Glu, Asn, Cys, His, Gln, Ser, and Thr. As disclosed in that application, however, it is important that the overall PAM width of the tree be considered before constructing hypotheses about the functional role of conserved residues.

III. Tools that Identify Individual Residues Involved in Changes in Functionally Significant Behavior.

In Ser. No. 08/914,375, it was disclosed that during episodes of rapid sequence evolution, amino acid substitutions will be concentrated in secondary structural elements. These are secondary structural elements that are important in the acquisition of new function. These elements might be predicted using the method claimed in Ser. No. 07/857,224; they might also be known by X-ray crystallography or n.m.r., for example. As n Ser. No. 08/914,375, a general method for identifying secondary structural elements that contribute to the origin of new biological function is comprised of identifying an element in the predicted secondary structure model where the corresponding section of the gene has a high ratio of expressed to silent changes.

In this analysis, we must recognize tthat function involves combinations of behaviors of a protein. Even when function changes, some features of those behaviors are conserved, and this reflects conservation of some features of the sequence as well. In the fumarase/aspartase/adenylosuccinate/argininosuccinate lyase example discussed elsewhere, all four proteins have the same overall fold. For this reason, residues critical to the folding process (for eample, amino acids whose side chains pack tightly into the folded core) will remain conserved even though the overall function of the protein is changing. Relevant to the change in function is, of course, a change in a number of behaviors, for example, the ability to bind a particular small molecule substrate. Residues involved in substrate binding will therefore be changing rapidly during the episode of sequence evolution where function was changing.

The notion that some residues are conserved even when function is chaning is matched by the notion that some residues will be changing even when function is conserved. The latter are those that can drift “neutrally”.

Likewise, “function” remains a concept set within Darwinian evolution. That is, a fumarase from a mesophile and a fumarase from a thermophile have analogous function in the sense that they both participate (for example) in the citric acid cycle. However, they have different functions, in that one contributes to fitness in a thermophile (which requires that it have an associated behavior, thermostability) while the other does not. In the epsidoe where the temperature of the environment changes, residues involved in conferring thermal stability will change, while those involved in determining substrate specificity will not.

Tools that assign, even at the level of hypothesis, which residues are involved in which behavior are extremely valuable. They can be the targets of protein engineering experiments, for example. In these cases, one would like to map residues identified using tools of the instant invention on to a three dimensional structure of a representative member of a protein family.

Already in 1988, the Applicant was using a general form of mapping that showed the utility of this in extracting information about the function of a protein, in this case, alcohol dehydrogenase [Ben89]. More recently, Lichtarge et al. introduced an evolutionary trace method that defined functionally significant residues as those that are conserved within a family [Lic96]. They then used this approach to identify patches on the surface of proteins that contribute to functionality.

As it was published, the evolutionary trace method was related to the method disclosed in Ser. No. 07/857,224, and was applied to conserve amino acid residues. The aproach did not contemplate the possibility that function might change within a family of proteins, and the residues important for function would change with it. Indeed, to detect such changes would require tools disclosed in this application and in Ser. No. 08/914,375 to be broadly useful.

A. Residues Changing in Episodes with High Kₐ/Kₛ Values, Minus Residues Changing in Episodes with low Kₐ/Kₛ Values

We have posited that function is changing during an episode with high Kₐ/Kₛ values. As disclosed in Ser. No. 08/914,375, individual residues can be identified as changing during that episode, as the basic evolutionary model has sequences reconstructed at each individual node. These are, at the level of hypothesis, residues that are important to functional change.

As one of ordinary skill in the art recognizes, the episode also includes a number of substitutions that have no relevance to function or the change in function, but rather reflect the background, neutral drift. For example, these residues might lie on the surface of the protein, be in contact with bulk solvent, and not have any especially strong functonal constraint that prevents them from diverging. As disclosed in Ser. No. 07/857,224, surface residues are likely to be neutrally drifing in many sub-families within an evolutionary tree. For this reason, we can identify residues that are changing along branches of an evolutionary tree that have low Kₐ/Kₛ values, and subtract them from residues changing in episodes with high Kₐ/Kₛ values. What remains are residues more likely, again at the level of hypothesis, to be involved in the change in function.

Ser. No. 07/857,224 disclosed and claimed methods for correlating changes in sequence with changes in the behavior of the protein. This in turn provides a method for identifying behavioral changes that are relevant to the change in function.

B. Residues Displaying Covarion Behavior

Again because the basic evolutionary model includes reconstructed ancestral intermediates, the methods of the instant invention identify specific residues that are displaying covarion behavior. These are residues that are under analogous functional constraints in different sub-families of the tree. This, in turn, implies that these particular residues contribute to a behavior that is conserved for a conserved feature of the function in distant branches of the tree.

C. Mapping These Residues on to Models for the Secondary, Tertiary, and Quaternary Structure of Proteins.

Insight into the relationship between function and amino acid sequence can be gained by mapping residues identified by Kₐ/Kₛ and covarion analysis onto a three dimensional structure. This identifies, for any particular branch, which residues are involved in changing function. This information is useful when attempting to identify residues that might be changed in a protein engineering experiment, for example.

IV. Tools that Identify Individual Residues Involved in Conserved of Functionally Significant Behavior

The type of analysis used for class II tools can also be applied to Class IV tools.

A. Residues Suffering Compensatory Changes

When a pair of residues suffers compensatory changes during a particular episode of protein sequence evolution, this implies that some physical property of the protein family must be the same at the end of the episode as it was at the beginning. This implies some conserved behavior important across that episode. The episode can, of course, be one where function in some sense is changing. Thus, in the fumarase/aspartase example mentioned above, one might identify residues the suffer compensatory changes during episodes where catalytic behavior is changing. These are residues most likely (at the level of hypothesis) to be important for folding, which is conserved over this episode. We can therefore use the methods of the instant invention to identify individual residues involved in conserved of functionally significant behavior

B. Sites Displaying Homoplasy

Sites that display homoplasy are subject to analogous functional constraints in different branches of the tree. Because of the evolutionary reconstructions in the basic evolutionary model, we know which positions they are are which amino acids involved. Therefore, we use the methods of the instant invention to identify individual residues involved in conserved of functionally significant behavior

C. Mapping These Residues on to Models for the Secondary, Tertiary, and Quaternary Structure of Proteins.

Insight into the relationship between function and amino acid sequence can be gained by mapping residues identified by Kₐ/Kₛ and covarion analysis onto a three dimensional structure. This identifies, for any particular branch, which residues are involved in changing function. This information is useful when attempting to identify residues that might be changed in a protein engineering experiment, for example.

V. Tools that Involve Correlation Between the Evolutionary Histories of Two Families of Proteins

Ser. No. 07/857,224 introduced in the first useful form the notion of compensatory changes as a way of analyzing divergent evolution in protein sequences. In that application, an example of compensatory covariation was identified that indicated the packing of two beta strands in an antiparallel fashion. A second use for compensatory changes disclosed was as part of a tool to detect disulfide bonds in a protein; cysteines that arise and/or disappear at the same time during the divergent evolution of a protein family frequently form a disulfide bond with each other. Ser. No. 08/914,375 extended this notion, noting that the introduction and loss of leptin and the leptin receptor might occur in parallel. The idea behind this analysis is that residues that interact as they contribute to function, subunits that interact as they contribute to function, and even proteins that interact as they contribute to function, display correlated evolution.

Since these applications were filed, various other groups have extended this approach. We review briefly two of the areas where research is active, and make comments on why additional invention is necessary to make these approaches fully useful

A. Correlating the Topology of Evolutionary Trees in Two Families of Proteins

Recently, Pellegrini et al. extended this type of analysis to generate “protein phylogenetic profiles” for different organisms [Pel99]. They present a method that assumed that during evolution, proteins that function together tend to be either preserved or eliminated in a new species. They described this property of correlated evolution by characterizing each protein by its phylogenetic profile, a string that encodes the presence or absence of a protein in every known genome. They suggested that proteins having matching or similar profiles strongly tend to be functionally linked. This method of phylogenetic profiling allows us to predict the function of uncharacterized proteins.

More recently, Cohen and his coworkers used phosphoglycerate kinase (PGK), an enzyme that forms its active site between its two domains, to develop a standard for measuring the co-evolution of interacting proteins. The N-terminal and C-terminal domains of PGK form the active site at their interface and are covalently linked. Therefore, they must have co-evolved to preserve enzyme function. By building two phylogenetic trees from multiple sequence alignments of each of the two domains of PGK, they calculated a correlation coefficient for the two trees that quantifies the co-evolution of the two domains. The correlation coefficient for the trees of the two domains of PGK is 0.79, which establishes an upper bound for the co-evolution of a protein domain with its binding partner. Their analysis was extended to ligands and their receptors, using the chemokines as a model [Goh00].

We have no quarrel with either of these approaches; indeed, they are in some ways covered by the Applicant's earlier disclosures. It should be recognized, however, that these simple approaches that exploit evolutionary analysis are easily defeated by the “ortholog paralog problem”, especially when it is coupled with gene loss. Briefly, paralogs are generated when a gene duplication occurs internally within a genome, to create two homologous genes in the same organism.

B. Correlating the Connectivity of Proteins in a Gene Family

Eisenberg and his coworkers [Enrxxx] and others have also suggested that proteins that interact in a pathway might be connected physically in the genome, either as an operon or, in some cases, in a single expressed polypeptide chain. This interesting approach is applicable to only a subset of the database, and is distinct from the tools disclosed here [Mar99].

C. Dating Events in the Molecular History

A key element to using evolutionary analysis of correlated change in protein families is to establish that the changes being interpreted as evidnce that two proteins interact as they function is to show that the changes are contemporaneous, that is, they occur near the same time. This requires tools that date, if only approximately, events in the molecular evolutionary tree using sequence data.

Early hope that protein sequences might change in a “clock-like” fashion [Can82], with a small number of rate constants describing the rate of change at most positions in most proteins in most organisms, has given way to the reality that the evolution of protein sequences is marked by episodes of rapid and slow evolution [Mes97]. These correspond to changing and conserved function within the protein family, arising in turn from adaptive and purifying natural selection, respectively. This makes methods based on protein sequence divergence unreliable for dating the divergence of protein sequences.

One well known approach to avoid (to a large extent, at least in metzoans) the influence of purifying and adaptive selection on the interpretation of molecular history is to examine changes in non-coding regions of DNA [Li97]. These include introns and substitutions, generally at the third position of a codon, that do not change the encoded amino acid. These arise because the genetic code is redundant for many amino acids. This approach assumes that silent substitutions at the DNA level have little or no impact on fitness (are neutral or nearly neutral) at the level of the organism. While this is almost certainly not a good approximation in microorganisms, the approximation appears to be serviceable for metazoans (multicellular animals) and plants, presumably because macrophysiology is more visible to selective forces than genome sequence itself in multicellular organisms.

Even silent substitutions are problematic as a molecular clock, however. From a chemical perspective, interconverting the four standard nucleobases A, G, T, and G involves 12 rate constants that need not be identical [Nei86]. Some models distinguish between transitions (purines replaced by purines, or pyrimidines replaced by pyrimidines) and transversions (purines replaced by pyrimidines, or pyrimidines replaced by purines), but otherwise group the rate processes together. This problem is revisited frequently in the literature [Nei86]. The most widely used method was developed by Li [Li85] with modifications by Pamilo and Bianchi [Pam93]. This method aggregates four fold redundant and two fold redundant sites, analyzes nucleotide substitution at positions where the encoded amino acid has not changed at the same time as it analyzes substitution at positions where the encoded amino acid has changed, and adopts a classification of different types of substitutions based on physical chemical characteristics of amino acids.

Disclosed here for the first time, the Applicant has discovered good part of the inconsistency in the dating generated by these methods can be eliminated if one focuses on relatively homogeneous chemical processes. In particular, transitions accumulate over large periods of (for example) vertebrate history with remarkable constancy, with a pseudo first order rate constant of 3.0×10⁻⁹ changes/base/year. A tool based on this discovery begins by extracting aligned pairs of codons from a pairwise alignment where two fold redundant amino acids (CDEFHKNQY) are conserved. Substitution at the silent position is then modelled using an exponential “approach to equilibrium” rate law, where f2 is the fraction of the codons encoding conserved 2FR amino acids that are themselves conserved: f2=[0.5·exp(−kt)]+0.5, where k is a single pseudo first order rate constant for transitions, and t is the time. The neutral evolutionary distance (NED) between two genes x and y is defined by NED_{x,y}=kt_{x,y}=−ln[(f2_{x,y}+0.5)/0.5].

NEDs represent one choice in a trade-off, between the instinct of a statistician (to maximize the number of characters being examined, and hence minimize error due to fluctuation) and the instinct of an organic chemist (to seek homogeneous rate processes, and hence minimize systematic error due to aggregation of different kinds of events).

The NED is a measure of evolutionary distance, not evolutionary time. If one knows the rate constant, and assumes that k is constant over the period of evolutionary history being examined, one can calculate the time of divergence. Given the same assumption and the date of evolutionary divergence of two sequences, one can calculate k. As distances, NEDs are additive, should obey the triangle inequality, and display other features that permit them to be used to build evolutionary trees.

The transition-based two fold NED turned out to be remarkably robust measures of evolutionary time. When calibrated using datable fossil divergences back to the divergence of fish from land vertebrates, a single lineage rate constant of 3×10⁻⁹ changes per base per year was obtained in many of the cases we examined, applicable (within error) to the divergence of fish from mammals, reptiles and birds from mammals, primates from artiodactyls, and artiodactyl genera from other artiodactyl genera. NEDs built from four fold redundant systems were far less consistent.

One of the key issues in the development of evolutionary models is assigning ranges of geological dates to nodes in the tree. Early hope that protein sequences might change in a “clock-like” fashion, with a small number of rate constants describing the rate of most amino acid substitutions in most proteins in most organisms, has given way to the reality that the evolution of protein sequences is marked by episodes of rapid and slow evolution. These correspond to changing and conserved function within the protein family, arising from adaptive and purifying natural selection respectively. This makes protein sequence similarity (for example, point accepted mutations per 100 amino acids, or PAM units) unreliable for dating the divergence of protein sequences.

One well known approach to avoid the influence of purifying and adaptive selection on the interpretation of molecular history is to examine changes in non-coding regions of DNA. These include introns and substitutions, generally at the third position of a codon, that do not change the encoded amino acid. These arise because the genetic code is redundant for many amino acids. Amino acids encoded by four synonymous codons (A₄'s) are valine, alanine, threonine, proline and glycine. Amino acids encoded by two synonymous codons (A₂'s) are cysteine, aspartic acid, glutamic acid, phenylalanine, histidine, lysine, asparagine, glutamine, and tyrosine. One amino acid (isoleucine) is encoded by three synonymous codons (A₃'s). These patterns are found in the eukaryotic nuclear code; other codes exist, of course.

This approach has a chance of working if silent substitutions at the DNA level have little or no impact on fitness at the level of the organism. While this is almost certainly not a good approximation in microorganisms (at least for some codons in highly expressed genes), the approximation appears to be serviceable for metazoans (multicellular animals), presumably because redundant codon exchange does not change the structure or the behavior of any functioning protein, and the structure and behavior of functioning proteins, together with the consequent macrophysiology, is more visible to selective forces than genome sequence itself. The approach is now empirically shown to be reliable within chordates.

Even silent substitutions are problematic as a molecular clock, however. From a chemical perspective, interconversion of the four standard nucleobases A, G, T, and G involves 12 rate constants that need not be identical (there is a large literature on this; see for example [Nei86]). Simpler models have distinguish between transitions (purines replaced by purines, or pyrimidines replaced by pyrimidines) and transversions (purines replaced by pyrimidines, or pyrimidines replaced by purines), but otherwise grouped the rate processes together.

This problem has been revisited frequently in the literature. The most widely used method (indeed, the one implemented in the present version of the Master Catalog when assigning Kₐ/Kₛ values, following some adaptations that we made, Schreiber, Benner unpublished) was developed by Li [Li85] with modifications by Pamilo and Bianchi [Pam93] following a suggestion by Kimura.

In the previous funding period, we developed and tested a NEDs as a tool for dating sequence divergences Table 1). NEDs turned out to be remarkably robust measures of evolutionary time. When calibrated using datable fossil divergences back to the divergence of fish from land vertebrates, a single lineage rate constant of 3×10⁻⁹ changes per base per year was obtained in many of the cases we examined, applicable (within error) to the divergence of fish from mammals, reptiles and birds from mammals, primates from artiodactyls, and artiodactyl genera from other artiodactyl genera. Statistical analysis suggests that >80% of the variance arises from simple statistical fluctuation. This suggests the absence of “hot spots” and other non-stochastic variation at the 2-fold degenerate sites in the genome. Again, relatively expensive tools (such as full blown ML tools) gave insignificantly different results than relatively cheap tools (such as the Pamilio-Bianchi approach) in a series of test cased that were applied in parallel.

TABLE
Average NED values for Pairs of Proteins Extraacted from Humans,
Pigs, Oxen, Rabbit, Rat, and Mouse

| | | | | Date | changes/base/year | |
|---|---|---|---|---|---|---|
| Species | Species | Number | kt (range) | (fossil) | k (calc.) | k (average) |
| 1 | 2 | of pairs | (NED) | MYA | × 10⁹ | × 10⁹ |
| Human | Pig | 225 | 0.3990 | 80 | 2.5 | |
| Human | Ox | 410 | 0.3800 | 80 | 2.4 | 2.4 |
| Pig | Ox | 140 | 0.2755 | 60 | 2.3 | |
| Rabbit | Human | 203 | 0.4845 | 80 | 3.0 | |
| Rat | Human | 584 | 0.4893 | 80 | 3.0 | 3.1 |
| Mouse | Ox | 147 | 0.5130 | 80 | 3.2 | |
| Mouse | Human | 918 | 0.4988 | 80 | 3.1 | |
| Mouse | Rabbit | 87 | 0.5083 | 60 | 4.2 | 5.2 |
| Mouse | Rat | 926 | 0.2470 | 20 | 6.2 | |

D. Correlating Evolutionary Events in Two Protein Families Occuring at Approximately the Same Time

Given approximate dates, we can now provide a more useful tool to correlate events occurring in two trees. A duplication in family 1 that is occurring near the time as a duplication occurring in family 2 is hypothesized to indicate that the two families (and, in particular, the proteins arising from the duplication) interact when they function. Conversely, and frequently quite usefully, a duplication in family 1 that did not occur near the time as a duplication occurring in family 2 is hypothesized to indicate that the two proteins arising from the duplication do not interact when they function. These hypotheses are ueful when designing two-hybrid systems, for example, to detect protein-protein contacts.

E. Correlating Evolutionary Events in Two Protein Families that are Associated with Analogous Behavior Involving Expressed/Silent Ratios

When there is a duplication, the question arises: Which of the derived genes is performing the derived function, and which is performing the ancestral function? According to the method of this invention, the derived protein is the one connected to the node where the duplication has occurred via the higher Kₐ/Kₛ value. This concept supports a useful tool to correlate events occurring in two trees. A duplication in family 1 that is occurring near the time as a duplication occurring in family 2 is hypothesized to indicate that the proteins arising from the duplication from the branch having the higher Kₐ/Kₛ value in one tree interact when they function with the proteins arising from the duplication from the branch having the higher Kₐ/Kₛ value in one tree interact when they function with the. Conversely, and frequently quite usefully, when examining two contemporarneous duplication events in two separate families, the proteins in family 1 that do not interact with the proteins in family 2 are those that are not joined to their respective nodes via branches that display, during contemporaneous periods of evolution, high Kₐ/Kₛ values.

As one of ordinary skill in the art will appreciate, this approach is quite general, and can be applied with covarion behavior, compensatory substitution, homoplasy, and even levels of high sequence conservation.

VI. Tools that Involve Correlation Between the Evolutionary History of a Family of Proteins and the Evolutionary History of the Organism as Known from Some Source Other than Genomic Sequence Data, Including Paleontology, Geology, Ecology, Ontogeny, Phylogeny, or Systematics (Collectively Known as the “Non-Genomic Record”.

The methods of this invention extract information about function and function change by analyzing sequence data alone, and then by coupling this analysis with secondary, tertiary, and quaternary structural data. Those of ordinary skill in the art know, of course, of other sources of evoluionary information that does not come from genomic sequence data or crystal structures. These “non-genomic” data come from paleontology, geology, ecology, ontogeny, phylogeny, and systematics (collectively known as the “non-genomic record”).

A. Correlating the Topology of an Evolutionary Trees and the Non-Genomic Record.

Conversely, and quite usefully, when a node in an evolutionary tree

Dates can be obtained approximately by protein sequence analysis. In cases where silent substitutions have not equilibrated, NED distances or other distances based on the analysis of silent codon substitutions can be used.

As discussed above, detailed analyses of evolutionary histories frequently can provide a solution to the most general problem of the conventional evolutionary paradigm, the difficulty in routinely identifying a homolog of a target sequence with known function within the database. By analysis of non-Markovian evolutionary behavior at the level of the protein, a model of secondary structure can be predicted. This prediction can be used in turn to detect long distance homologs in some cases and exclude the possibility of distant homology in others. This increases the likelihood that a homolog will be found with a known structure, behavior, or function for a new protein sequence. If one is found, then the logic associated with the conventional evolutionary paradigm can be applied to generate a hypothesis concerning the behavior or function of the protein.

The value of this post-genomic tool to assign behavior and structure to a target sequence problem is expected to grow over the near term, as the ratio of sequences supported by experimental studies to those not supported increases with the conclusion of genome projects, and as more sequences increase the detail of the evolutionary histories that can be extracted from the database directly, and therefore the quality of the predicted secondary structural model.

At the next level, analysis of non-Markovian behavior at the level of the gene can alert the biological chemist that the logic associated with the conventional evolutionary paradigm might not apply in individual cases. In particular, if an episode of rapid sequence evolution intervenes in the evolutionary tree between the sequence of interest and the sequence with the know behavior and function, the biological chemist is alerted to the possibility that the function of the protein might have changed. This alert is useful even with close homologs, as illustrated in the example with leptin.

But what if the evolutionary tree contains no protein with a sequence with assigned function, even one with low sequence similarity? Even with more limited evolutionary histories, post-genomic tools that analyze non-Markovian evolution at the level of the codon can be useful. By identifying the organisms that provide the sequences at the “leaves” of the evolutionary tree, it is frequently possible to correlate branches in the evolutionary tree with episodes in geological history, as determined from the fossil record. Especially in multicellular animals (metazoa), the fossil record can provide approximate dates for the emergence of new physiological function. In this case, it is possible to ask whether an episode of rapid sequence evolution in a protein family (in particular, an episode with a high expressed/silent ratio) occurred at the same time as a new physiological function emerged on earth. If so, a first level of hypothesis about physiological function can be proposed, even if no behavior or function of any kind is known for any of the modern proteins.

Perhaps the most transparent analysis of this type concerns proteins that underwent massive radiative divergences in metazoa approximately 600 million years ago. This is the time of the Cambrian explosion, an episode in terrestrial history that marks the massive radiative divergence of multicellular animals, including chordates. Proteins families undergoing rapid evolution at this time (for example, of protein tyrosine kinases and src homology 2 domains) are almost certainly involved in the basic processes by which multicellular animals develop from a single fertilized egg.

This type of analysis might be applied in the family of ribonuclease (RNase) A (E.C.2.7.7.16), a well known family of digestive proteins found in ruminants. The protein underwent rapid sequence evolution approximately 45 million years ago, a time where ruminant digestion emerged in mammals [Jer95]. Thus, the rapid molecular evolution evident in the reconstructed evolutionary history of this protein suggests that the protein is important for ruminant digestive function.

B. Correlating Features of Patterns of Evolution in Specific Branches in the Evolutionary Tree with the Non-Genomic Record

This type of analysis is obviously strengthened if one adds now information concerning Kₐ/Kₛ values, covarion behavior, homoplasy, and compensatory changes.

C. Correlating Evolutionary Events in Several Protein Families Occuring at Approximately the Same Time with the Non-Genomic Record

This type of analysis can obviously contribute to the determination of pathways, interactions between proteins from different families. These hypotheses are ueful when designing two-hybrid systems, for example, to detect protein-protein contacts.

Use of Non-Stochastic Behavior Generally

One of ordinary skill in the art will recognize from Ser. No. 07/857,224 that the methods of the instant invention view molecular evolution in a way quite distinct from the way in which standard tools analyze protein sequence data. Virtually all tools for comparing the sequences of homologous proteins assume a model for divergent evolution that is stochastic in outcome. This model treats a protein sequence as a linear string of letters, one letter for each amino acid. According to the model, each letter in the string changes (the gene and its corresponding protein mutates) at a rate that is independent of its position. According to the stochastic model, future and past mutations are independent. Mutations at one position are independent of mutations elsewhere.

Such a model is at best an approximation for the reality of protein evolution. In reality, proteins are not linear strings of letters. Rather, they are organic molecules that fold in three dimensions. In the folded form, some positions in a protein sequence are more easily mutatable (without destroying function) than others. Amino acids distant in the sequence but close in the fold frequently undergo correlated mutation. Future mutations are frequently not independent of past mutations. Thus, real proteins divergently evolving under functional constraints behave differently than expected based on the stochastic model.

The difference between the reality of divergent evolution of proteins that fold and expectation based on the stochastic model proves to be important, as was disclosed first in Ser. No. 07/857,224. By comparing the patterns of substitution within a set of folded proteins undergoing divergent evolution with expectations for those patterns based on the stochastic model, one can extract information about the fold. This makes the nuclear family more than a database organizational feature. Because the nuclear family holds a history of the pattern of divergent evolution under functional constraints in the protein, it holds information about the fold of the protein. From the sequences of proteins in the nuclear family alone, one can decide which amino acids lie on the surface of the folded structure, which lie inside, and which lie near the active site. Elements of secondary structure, the helices, strands, and loops can be identified. A model of tertiary structure can be built as well, all from the evolutionary history embodied in the nuclear family.

### EXAMPLES

### Example 1

### Functional Analysis of Aromatase

Aromatase is a cytochrome P450-dependent enzyme that catalyzes a three step reaction that creates an estrogen from an androgen. The physiological consequences of estrogen biosynthesis in human biology are well known, even among laymen. Estrogen is also synthesized in primitive chordates such as Amphioxus (Callard et al., 1984), but not in other metazoans. Therefore, estrogen appears to have been invented as a hormone early in the divergent evolution of chordates, presumably by recruitment of steroids involved in developmental biology in more primitive metazoan ancestors.

Aromatase belongs to the cytochrome P450 superfamily of enzymes, which has some two dozen family members (Nebert et al., 1991). Members of the superfamily use a common chemical mechanism (Akhtar et al, 1997) to assimilate carbon, detoxify organic substances, and synthesize regulatory molecules. In biomedicine, variants of P450 oxidases can determine whether individuals have side effects to a therapeutic agent (Gonzalez & Nebert, 1990), and aromatase itself plays a significant role in the progression of some cancers.

Recent research has found remarkable complexity in the molecular biology of the aromatase gene family. Two aromatase genes are known in goldfish [Cal97]. In contrast, only a single gene is known in the horse [Boe97], the rat [Hic90], the mouse [Ter91], the human [Har88], and the rabbit [Del96]. Both a functional gene and a pseudogene are found in oxen. The pseudogene is built from homologs of exons 2, 3, 5, 8, and 9 interspersed with a bovine repeat element [Fue95] it is transcribed but not translated. In several mammalian species, a single gene yields multiple forms of the mRNA for aromatase in different tissues via alternative splicing mechanisms. This is the case in humans [Sim07] and rabbits [Del98].

A still different phenomenology is observed in the pig (Sus scrofa). Preliminary studies found three distinct mRNA molecules in different tissues with differences in their coding regions [Con96][Con97][Cho96][Cho97a][Cho97b]. It was suggested that these might have arisen from a single gene, possibly via RNA editing or alternative splicing.

Analogous collections of phenomenology are found throughout contemporary molecular biology for many molecular systems. “Why?” questions are often confounded by the complexity of the phenomenology. When “just so” stories are proposed, they need not be compelling, especially when they are supported by no evidence past the phenomena themselves.

One approach to obtain additional evidence to address functional questions in systems requires placing the molecular biological phenomena within an evolutionary context. To do this for the aromatases family, we began with experiments to determine whether the three mRNA isoforms (and the corresponding proteins) in pig arose through alternative splicing, via mRNA editing, or from distinct genes. PCR primers were designed from sequences located within the previously characterized exon 4 of the porcine aromatase type III gene [Cho96][Cho97a], a region that the cDNA studies suggested might have internal sequence differences [Choi97][Con97] and used to amplify pig genomic DNA. Initially, eight clones of the PCR products were sequenced. Four of these had the sequence corresponding to aromatase isoform I (ovarian type) as identified from cDNA, while four others had the sequence corresponding to aromatase isoform III (embryo type) as identified from cDNA.

With evidence that at least two aromatase genes could be found in pig genomic DNA, a restriction enzyme-based assay was designed to search genomic DNA in greater detail. Nsi I digests exon 4 from isoform I twice, and isoform III once. Bsm I digests exon 4 from isoform I once, but not exon 4 of isoform III. Exon 4 from isoform II (placental type) had no restriction sites for either enzyme. Restriction analysis of a total of 23 clones obtained from genomic DNA identified 8, 5, and 10 representatives of isoforms I, II, and III, respectively. No restriction digestion patterns indicative of a novel sequence were observed. Representative clones for isoforms I, II, and III were then sequenced. To further confirm the presence of exactly three aromatase isoforms within the porcine genome, primer pairs were designed from within the 5′ and 3′ junctions of exon 7. Sequence analysis of 10 clones derived from the PCR products identified six and four clones of isoforms II and III, respectively

With compelling evidence that the three variants of mRNA identified in cDNA studies arose from three paralogous genes (as opposed to editing or alternative splicing), we sought to place the paralogous genes within their historical context. Following standard tools to analyze protein sequences, pairwise alignments were constructed for the 136 pairs of proteins. An evolutionary distance (in PAM units) was calculated (with a variance) for each pair (Table 1). From this, an evolutionary tree was built for the mammalian sequences (Drawing 4), with branch lengths along internal nodes calculated to minimize a least squares distance were then constructed within the Darwin programming environment. The tree was adjusted to make the human and equine branchings consistent with paleontological records to obtain a “best consensus” tree. The sequences of the ancestral genes and proteins at branch points in the tree were then reconstructed. From there, mutations (including fractional mutations) at both the DNA level and protein level were assigned to individual branches in the tree using the method of Fitch [Fit91].

Based on the tree and the reconstructed evolutionary intermediates, Kₐ/Kₛ values were assigned to individual branches using the method of Li et al. (1985). These reflect the normalized ratio of substitutions at the level of the gene that change the encoded polypeptide sequence (non-synonymous substitutions) to substitutions at the level of the gene that do not change the encoded polypeptide sequence (synonymous substitutions). Lower Kₐ/Kₛ values generally reflect conservative episodes of evolution where function remains constant, while higher values frequently characterize episodes of evolution where function is changing [Tra96][Mes97].

The average branch in the aromatase evolutionary tree has a value of Kₐ/Kₛ of 0.348. Inspection of the tree shows that the highest Kₐ/Kₛ values anywhere in the mammalian aromatase family (0.85 and 0.66) are found within the divergent evolution of the pig aromatases. These suggest that adaptive changes occurred during the triplication of the aromatase gene in pigs. Adaptive changes are well known to confuse simple models of molecular history built from standard sequence alignment and tree construction tools. Adaptive substitutions do not conform to stochastic rules modelling divergent evolution [Ben97], do not accumulate in a clock-like fashion, and may arise through convergent and parallel evolution [Ste87].

Therefore, the evolutionary history of the aromatase family was re-analyzed using pairwise Neutral Evolutionary Distances (NEDs), obtained for the 136 pairs of aligned aromatase genes (Table 2). To estimate NEDs between the aromatase gene pairs, the number (n) of “2-fold redundant amino acids” (Cys, Asp, Glu, Phe, His, Lys, Asn, Gln, and Tyr) that are conserved in the aligned pairs was determined. The number of those amino acids that are encoded by the same codon (c) was then determined, and the fraction ([f2=c/n) of the codons that are the same is then tabulated (Table 2).

A variety of empirical studies show that the fixation of silent substitutions in conserved 2-fold redundant codon systems follows rate law that is a simple exponential “approach to equilibrium” f2=[0.5·exp(−kt)]+0.5, where k is a single pseudo first order rate constant for transitions, and t is the time [Juk69]. The NED distance is defined by NED_{x,y}=kt_{x,y}=ln[(f2_{x,y}+0.5)/0.5].

The NED is a measure of evolutionary distance, not of evolutionary time. As distances, NEDs are additive, should obey the triangle inequality, and display other features that permit them to be used to build evolutionary trees, provided that k is constant over the period of evolutionary history being examined. A variety of empirical studies shows this to be approximately the case for many protein families. The approximation appears to be quite good for aromatase as well. Thus, if a fixed single lineage first order rate constant of 3×10⁻⁹ changes per base per year is assumed, the NED values indicate that fish and land vertebrates diverged 340 million years ago (mya), birds and mammals diverged 250 mya, primates and ungulates diverged 73 mya, horse and artiodactyls diverged 71 mya, and pigs and ruminants diverged 62 mya. Each of these dates is close to the date suggested by the paleontological record [Car88].

The NED-based dating was used to assess two alternative models to explain the triplication of aromatase gene family in pigs. The first, advanced by Callard and Tchoudakova [Cal97], holds that the physiological specialization of aromatases through the formation of paralogs occurred early in vertebrate divergence, perhaps 400 mya, before fish and mammals diverged. If this were the case, then a functional explanation for the aromatase genes must be sought in fundamental features of vertebrate developmental biology, those that emerged early in vertebrate evolution. Conversely, the triplication of aromatase may occur in response to the domestication of pigs. In this case, a functional explanation for the aromatase genes would be found in the selective pressures applied by breeding programs.

The NEDs separating the three pig isoforms range from 0.154 (corresponding to a distance of 51 million years between the proteins) to 0.199 (corresponding to a distance of 66 million years). Recognizing that the total distances between two proteins are twice the distance along a single lineage from the point of divergence to the modern protein (half of the distance occurrs along one lineage after divergence, and half of the distance occurs along the other lineage), the NEDs suggest that the first duplication led to the three porcine aromatase genes occurred ca. 33 mya, and the second occurred ca. 25 mya. An evolutionary tree constructed from these NEDs is consistent with these conclusions, showing that the porcine aromatases branched after the lineage leading to pig diverged from the lineage leading to ox (Drawing 5). This tree shows a different branching order for the three porcine paralogs than the tree based on amino acid sequences, something not uncommon in the presence of substantial adaptive evolution. Nevertheless, the data are consistent with an evolutionary model that holds that the ancestor of pig and oxen (approximated in the fossil record most closely by the now extinct Diacodexis which lived perhaps 55 mya) contained a single aromatase gene, and that the paralogous genes in pig arose ca. 25 million years later. Thus, the paralogs in pig can be explained neither in terms of the fundamentals of vertebrate development, nor as a consequence of swine domestication.

Error in these dates can arise from two sources, standard error (which arises from fluctuation) and systematic error (which arises from the fact that the evolutionary model does not represent actual evolution). The first can be calculated by standard statistical approaches using standard statistical assumptions. The second cannot be calculated, as too little is known about possible systematic errors in the evolutionary model. The f2 distances are each based on ca. 120 two-fold redundant codon systems, and variances for the NEDs are given in Table 2. Inspection of the tree in Drawing 5 gives an indication of the actual error, as the NED between any ancestral sequences and all modern sequences derived from it should be the same. The calculated distance from the divergence of the three porcine enzymes to the type II enzyme is 31 million years, to isoform I is 32 million years, and to isoform III is 30 million years. Thus, the average reported (31 mya) could be as low as 30 and as high as 32 mya. All of these dates are in the Oligocene, after the first episode of cooling. The divergence of isoform I and III ranges from 24-26 mya. These apparent errors are less than the errors associated with the dating (from the fossil record) used to set the molecular clock.

Instead, an understanding of why pigs have three genes for aromatase must lie in the environment of (and events that occurred during) a time on Earth 25-33 mya. For this we turn to the paleontological, paleogeographical, and paleoclimatological records of that period, which is near the boundary between the Oligocene (38-25 mya) and the Miocene (25-5 mya), two epochs in the Cenozoic “Age of Mammals” [Pro94]. This period is an unusual one in the history of the Earth. When characterized globally, the Earth during the Eocene (54-38 mya) was warm and tropical, evidently free of ice over the entire planet. By the end of the Eocene, however, the Earth had begun to suffer a dramatic cooling that was to lower the mean annual temperature by as much as 15° C. [Wol98]. Areas of the planet became covered with ice. And the impact of the cooling on the biosphere was dramatic. For example, perhaps 80% of the North American faunal genera became extinct ([Pro94] pp 113-114) [Stu90]. By the end of the Oligocene and into the Miocene 25 mya, however, the global cooling abated, the climate turned warmer, and the biosphere became more tropical.

Did this climate change occur in the environment where the ancestors of modern pigs were living just before the Oligocene-Miocene boundary? At this time, the North American and Eurasian fauna were geographically isolated. Modern peccaries (Tayassuidae), not pigs, emerged in the New World from ancestral suids that immigrated from Asia. North America cannot be the site for the triplication of the aromatase genes in pig, therefore, and its climate 25-33 mya is irrelevant to an explanation for the triplication of the aromatase genes in pigs.

Instead, modern pigs most likely emerged in Europe near the end of the Oligocene [Coo78] [Pil91] from more primitive entelodonts such as Archaeotherium. During the Oligocene, the Dichobunids (the most probable ancestral stock) were most abundant in Europe. Likewise, the first true pig, Propalaeochoerus, from the late Oligocene, was common only in Europe [Coo78][Car88]. This makes the paleoenvironment of Europe near the Oligocene-Miocene boundary relevant to the functional implications of the aromatase gene triplication in pigs.

Various paleobiological evidence suggests that the climate in Europe also deteriorated in the Oligocene and warmed in the Miocene. A study of amphibian distribution in the Oligocene of Europe, for example, is consistent with a significant drop of mean annual temperatures in the European Oligocene. In the Miocene, amphibians populations rebounded, corresponding to an improvement in the climate [Roc96]. Likewise, analysis of the deer population suggested a subtropical climate returning to Europe in the early Miocene [Anz93]. The Iberian peninsula in the early Miocene had an intertropical to subtropical climate [Mur99]. Crocodiles also returned to Europe at the Oligocene-Miocene boundary [Ant99]. The presence of arboreal primates in the European Miocene also suggests a forested environment [Qi98]. Each of these facts (and many others) suggests that the second duplication of the aromatase gene in pigs occurred at the same time as the return of subtropical and warm temperate forests and woodlands to Europe, the type of environment for which suids are best adapted [For96].

Immediately thereafter, the suids underwent a significant radiative divergence, and came to occupy all of the Old World. By the early Miocene, the two basal members that were to lead to all modern pigs, Hyotherium and Xenochoerus, were widespread in Europe, Asia, and Africa. The amelioration of the climate evidently assisted in this spread. For example, the pigs now in Africa apparently came from southwest Asia in the Early Miocene. A fossil of this date of a tetraconodontine pig has been reported from the Levant [van99], through which the pigs would have migrated to get from Eurasia to Africa, and which was a tropical environment at the beginning of the Miocene [Tch92]. In the middle and late Miocene, modern suids had diversified in Europe in further response to the change in the paleoclimate [For96].

Why might a change in climate with a return of forested (and perhaps tropical) ecosystems have led to a selection of pigs that had three different aromatase genes? We turned to porcine reproductive physiology for insight. We recently found that the type III aromatase was expressed by the embryo between day 11 and day 13 following fertilization, during the late pre-implantation period [Cho97a,b]. The estrogen generated by the type III isoform causes uterine undulation. This undulation, in turn, is expected to cause the spacing of the ca. 30 eggs that are fertilized in a typical conception, which eventually yield the 8-12 piglets that are normally birthed. In pigs, if the litter does not contain at least 5 individuals, the entire conception is aborted. Thus, the embryonic form of aromatase may have a role in spacing the embryos uniformly around the uterus, and preventing abortion. These are useful adaptations if one wants to have an increased litter size.

Evidence in the paleontological record suggests that the size of the litter in pigs increased dramatically 25-30 mya, at the same time as isoform III of aromatase was generated by triplication, the local paleoclimate warmed, and the pigs began a major radiative divergence. The ancestral suid Archaeotherium, disappearing from the fossil record at the end of the Oligocene, may have given birth to a single pup. All of the contemporary forms of pigs arising from the divergence of Hyotherium and Xenochoerus, known from the Early Miocene, have large litter sizes. Further, Archaeomeryx, the early Eocene artiodactyl that is presumed to be the ancestral ruminant, resembles the contemporary chevrotain, which also births a single pup.

The biogeography of the suids was again consulted to test the hypothesis that litter size increased in the suids near the time that the climate changed and the aromatase gene triplicated. As noted above, peccaries were isolated in the New World in the Early Oligocene, before the NED-derived date for the triplication of the aromatase gene in the Old World pigs. Consistent with the model, the peccary has only one offspring. The model predicts as well that the peccary should have only a single aromatase gene.

| | | |
|---|---|---|
| Pig | | |
| Type | I | C AAT CAT TAC ACG TGC CGA TTT GGC AGC AAA |
| | | CTT GGG TTG GAA |
| | | N H Y T C R F G S K |
| | | L G L E |
| | III | T AGT CAC TAC ACA TCC CGA TTT GGC AGC AAA |
| | | CCT GGG TTG CAG |
| | | S H Y T S R F G S K |
| | | P G L Q |
| | II | C AGT CAC TAC ACA TCC CGA TTC GGC AGC AAA |
| | | CCT GGG TTG GAG |
| | | S H Y T S R F G S K |
| | | P G L E |

| | |
|---|---|
| Peccary | C AGT CAC TAC ACA TCC CGA TTC GGC AGC AAA |
| | CCT GGG TTG CAG |
| | S H Y T S R F G S K |
| | P G L Q |

| | | |
|---|---|---|
| Pig | | |
| Type | I | TGC ATT GGC ATG CAT GAA AAA GGC ATC ATG |
| | | TTT AAC AAT AA |
| | | C I G M H E K G I M |
| | | F N N N |
| | III | TTC ATT GGC ATG CAT GAG AAA GGC ATT ATA |
| | | TTC AAC AAT AA |
| | | F I G M H E K G I I |
| | | F N N N |
| | II | TGC ATC GGC ATG TAT GAG AAG GGC ATC ATA |
| | | TTT AAT AAT GA |
| | | C I G M Y E K G I I |
| | | F N N D |

| | |
|---|---|
| Peccary | TTC ATT GGA ATG CAT GAG AAA GGC ATC ATA |
| | TTT AAC AAC AA |
| | F I G M H E K G I I |
| | F N N N |

To test this prediction, peccary seminal plasma (from the Center for Reproduction of Endangered Species, Zoological Society of San Diego) was subjected to PCR amplification using exon 4-specific primers as described above. Bands having the expected sizes were observed by agarose gel electrophoresis. Five clones derived from the PCR products were found to have identical sequences, all different from the sequences of the pig aromatase. The NED comparison (using a rate constant of 3×10⁻⁹ changes per base per year) suggested that the peccary diverged 40 mya from the pig, corresponding to the fossil record and the known isolation of the New and Old World paleoecosystems.

The molecular biological, fossil, paleoecological, and physiological evidence are all consistent with a model that proposes that climate changes in Europe at the end of the Oligocene selected for pigs that had larger litter sizes. The successful lineage generated a new embryo aromatase by gene duplication, and expressed it at the time of implantation, forming the molecular basis of the physiology that enabled large litter sizes. It is possible to speculate on why a conversion from an open, savannah like environment to a forested environment might enable larger litter sizes. Contemporary savannah babies are large and born with the ability to run, presumably because hiding is no alternative. In contrast, in a forested environment, pups are easier to hide, permitting them to be smaller and less precocious at birth, permitting in turn a larger number of pups for the same total birth weight. Indeed, the contemporary Sus scrofa sow hides her piglets in earthen hollows covered with leaves [Eis81].

Implantation is one of the least well understood steps in mammalian reproductive biology, including human reproductive biology. Implantation is, of course, found only in mammal reproductive physiology, and is itself therefore a relatively recent innovation in physiology, emerging perhaps 200 million years ago. This analysis emphasizes the degree of innovation and experimentation that is continuing in mammalian reproductive physiology. Further, the analysis is a combination of computational informatics, geology, paleontology, physiology, molecular biology and chemistry. Analogous analyses should be applicable in functional genomics throughout the biological, biomedical and biochemical sciences, especially as genome projects are completed and as new tools become available to analyze genomic databases.

### Example 2

### Covarion Behavior in Alcohol Dehydrogenases

Mammalian alcohol dehydrogenase (E.C.1.1.1.1) have undergone a rapid episode of sequence evolution in and around the active site as substrate specificity has divergently evolved to handle xenobiotic substances in the liver. In contrast, over a comparable span of evolutionary distance, the active site of yeast alcohol dehydrogenase has changed very little, corresponding to an apparently constant role of the enzyme to act on the ethanol-acetaldehyde redox couple. Indeed, by identifying positions in mammalian dehydrogenases where amino acid variation was observed over a span of evolution where the same residues were conserved in the yeast dehydrogenases provided a clear map of the active site of the protein [Ben88].

### Example 3

### Identifying Mutations and In Vitro Properties of Seminal Ribonuclease that Contribute to Selected Function.

Bovine seminal ribonuclease (RNase) diverged from bovine pancreatic RNase approximately 35 million years ago. Seminal RNase represents approximately 2% of the total protein in bovine seminal plasma. It displays antispermatogenic activity [Dos73], immunosuppressive activity [Sou81] [Sou83] [Sou86], and cytostatic activity against many transformed cell lines [Mat73] [Ves80]. Each of these biological activities is essentially absent from pancreatic RNase. Further, seminal RNase binds to anionic glycolipids, binds and melts duplex DNA, hydrolyzes duplex RNA, has a dimeric quaternary structure, and binds to spermatozoa.

Each of these behaviors is measured in vitro and is well known in the art. In the absence of the method of the instant invention, the behaviors are difficult to interpret. Some, any, or all of the behaviors might serve an adaptive role. It is possible that none of these behaviors serve adaptive roles. Indeed, it is conceivable that the protein has no adaptive role at all. This makes it difficult to make even the simplest research decisions, as the only in vitro properties of a protein that are interesting to study are those that have a physiological function.

To resolve these issues, genes for seminal and pancreatic RNases were obtained from a variety of organisms closely related to Bos taurus, using cloning procedures well known in the art. These were then sequenced, and a maximum parsimony tree was constructed using MacClade. From this tree were calculated the sequences of RNases that were intermediates in the evolution of the seminal RNase, using the maximum parsimony method well known in the art.

Next, the ratio of expressed to silent substitutions was calculated along each branch of the evolutionary tree. A very high ratio of expressed to silent substitutions was observed in the evolutionary period following the divergence of kudu [Tra96] from the lineage leading to ox, until the divergence of water buffalo and ox. This is indicative of an episode of adaptive evolution, where the protein acquires a new physiological function. Further work indicated that the seminal RNase gene was not expressed in the period of evolution since the divergence of the seminal RNase family and the divergence of kudu.

Last, protein engineering methods were used to prepare the seminal RNase that was at the beginning of the episode of rapid sequence evolution. It properties were then examined experimentally. It was discovered that the ability of the protein to bind to anionic glycolipids was roughly the same before and after this episode of rapid evolution. So too was its sensitivity to inhibition by placental RNase inhibitor. Thus, both of these properties are not likely to be under selective pressure.

In contrast, the immunosuppressivity of the ancestral RNase (IC₅₀ ca. 8 micrograms/mL) was greater than that of pancreatic RNase (IC₅₀ ca. 100 micrograms/mL). But following the period of rapid sequence evolution characteristic of a protein evolving to serve a new physiological function, the immunosuppressivity became still greater (IC₅₀ ca. 2 micrograms/mL). Thus, one concludes that immunosuppressivity as measured in vitro is a selected trait of the protein, or is closely structurally coupled to a trait that is selected.

Likewise, the ability of the seminal RNase protein to bind and melt duplex DNA, and to hydrolyze duplex RNA, also underwent rapid increase between the time of divergence of kudu from modern ox. Thus, it too is either a selected trait of the protein, or is closely structurally coupled to a trait that is selected.

In vitro experiments in biological chemistry extract data on proteins and nucleic acids (for example) that are removed from their native environment, often in pure or purified states. While isolation and purification of molecules and molecular aggregates from biological systems is an essential part of contemporary biological research, the fact that the data are obtained in a non-native environment raises questions concerning their physiological relevance. Properties of biological systems determined in vitro need not correspond to those in vivo, and properties determined in vitro need have no biological relevance in vivo.

To date, there has been no simple way to say whether or not biological behaviors are important physiologically to a host organism. Even in those cases where a relatively strong case can be made for physiological relevance (for example, for enzymes that catalyze steps in primary metabolism), it has proven to be difficult to decide whether individual properties of that enzymes (k_{cat}, Kₘ, kinetic order, stereospecificity, etc.) have physiological relevance. Especially difficult, however, is to ascertain which behaviors measures in vitro play roles in “higher” function in metazoa, including digestion, development, regulation, reproduction, and complex behavior.

Analysis of non-Markovian behavior, as described above, permits the biological chemist to identify episodes in the history of a protein family where new function is emerging. This suggests a general method to determine whether a behavior measured in vitro is important to the evolution of new physiological function. We may take the following steps:

(a) Prepare in the laboratory proteins that have the reconstructed sequences corresponding to the ancestral proteins before, during, and after the evolution of new biological function, as revealed by an episode of high expressed to silent ratio of substitution in a protein. This high ratio compels the conclusion that the protein itself serves a physiological role, one that is changing during the period of rapid non-Markovian sequence evolution.

(b) Measure in the laboratory the behavior in question in ancestral proteins before, during, and after the evolution of new biological function, as revealed by an episode of high expressed to silent ratio of substitution. Those behaviors that increase during this episode are deduced to be important for physiological function. Those that do not are not.

Each of the behaviors displayed by seminal RNase is measured in vitro, as is the case for a wide range of biological phenomenology recorded in the literature. The behaviors are difficult to interpret. Some, any, or all of the behaviors might serve an adaptive role. It is possible that none of these behaviors serve adaptive roles. Indeed, it is conceivable that the protein has no adaptive role at all. This makes it difficult to make even the simplest research decisions, as the only in vitro properties of a protein that are interesting to study are those that have a physiological function.

To resolve these issues using the post-genomic method outlined above, genes for seminal and pancreatic RNases were obtained from a variety of organisms closely related to Bos taurus, using cloning procedures well known in the art. These were then sequenced, and a maximum parsimony tree was constructed using MacClade. From this tree were calculated the sequences of RNases that were intermediates in the evolution of the seminal RNase, using the maximum parsimony method and checked using maximum likelihood tools implemented in Darwin.

Next, the ratio of expressed to silent substitutions was calculated along each branch of the evolutionary tree. A very high ratio of expressed to silent substitutions was observed in the evolutionary period following the divergence of cape buffalo [Tra96] from the lineage leading to ox, until the divergence of water buffalo and ox. This is indicative of an episode of adaptive evolution, where the protein acquires a new physiological function. Further work indicated that the seminal RNase gene was not expressed in the period of evolution since the divergence of the seminal RNase family and the divergence of cape buffalo.

Last, protein engineering methods were used to prepare the seminal RNase that existed at the beginning of the episode of rapid sequence evolution. Its properties were then examined experimentally. It was discovered that the ability of the protein to bind to anionic glycolipids was roughly the same before and after this episode of rapid evolution. So too was its sensitivity to inhibition by placental RNase inhibitor. Thus, both of these properties are not likely to be under selective pressure.

In contrast, the immunosuppressivity of the ancestral RNase (IC₅₀ ca. 8 micrograms/mL) was greater than that of pancreatic RNase (IC₅₀ ca. 100 micrograms/mL) (J. Sleasman, M. Rojas, personal communication). But following the period of rapid sequence evolution characteristic of a protein evolving to serve a new physiological function, the immunosuppressivity became still greater (IC₅₀ ca. 2 micrograms/mL). Thus, one concludes that immunosuppressivity as measured in vitro is a selected trait of the protein, or is closely structurally coupled to a trait that is selected.

Likewise, the ability of the seminal RNase protein to bind and melt duplex DNA, and to hydrolyze duplex RNA, also underwent rapid increases between the time of divergence of cape buffalo from modern ox. Thus, it too is either a selected trait of the protein, or is closely structurally coupled to a trait that is selected. In contrast, dimeric structure did not emerge during this period. Dimeric structure, therefore, is presumably not as important to the new selected function of the protein, although it may be a trait that was initially useful in the selection of the system for further optimization during the period of rapid evolution.

### Example 4

### Assignment of Episodes of Adaptive Evolution in the Protein Leptin, and Placing These in Predicted Secondary Structural Elements

From the GenBank database, DNA and protein sequences were retrieved for the genes encoding leptins and the corresponding proteins, also known as the obesity gene product. A multiple alignment for the protein sequences was constructed for the DNA sequences and the protein sequences. These were converted to a file suitable for MacClade to use. For both the DNA and protein sequences, a tree using MacClade was built based on the known relationship between the organisms from which these sequences were derived; this proved to be the most parsimonious tree as well. MacClade was also used to built a tree for the protein sequences based on the known relationship between organisms; this proved not to be the most parsimonious tree (by 1 change). The DNA tree was taken to be definitive because of its consistency with the biological (cladistic) data showing that the primates form a clade.

A secondary structure prediction was made for the protein family using the tools disclosed in Ser. No. 07/857,224. The evolutionary divergence of the sequences available for the leptin family is small; only 21 PAM units (point accepted mutations per 100 amino acids), predictions were biased to favor surface assignments [Ben94]. Thus, positions holding conserved KREND were assigned as surface residues, conserved H and Q were assigned to the surface as well, while positions holding conserved CST were assigned as uncertain. suface and interior assignments are summarized in Table 3.

A secondary structure was then predicted for the leptins using the methods disclosed in Ser. No. 07/857,224. The multiple alignment is shown in Table 3. Five separate secondary structural elements were identified results are summarized in Table 3. A disulfide bond is presumed to connect positions 96 and 146. These secondary structural elements can be accommodated by only a small number of overall folds. Interestingly, the pattern of secondary structure in this prediction is consistent with an overall fold that resembles that seen in cytokines such as colony stimulating factor and human growth hormone [deV92].

To decide whether evolutionary function may have changed under selective pressure during the divergent evolution of the protein family, a multiple alignment of the protein sequences and a multiple alignment for the corresponding DNA sequences were constructed. A MacClade-generated maximum parsimony tree was printed for each position in the protein sequence where there was a change, and for each position in the DNA sequence where there was a change. Each mutation on each tree was examined by hand, and silent and expressed mutations occurred were assigned to individual branches on the evolutionary tree. For each branch of the tree, the sum of the number of silent and expressed changes were tabulated, and the ratio of expressed to silent changes calculated. These are shown in Drawing 1. Tables 4 and 5 contain the data used in this example.

The branches on the evolutionary tree leading to the primate leptins from their ancestors at the time that rodents and primates diverged had an extremely high ratio of expressed to silent changes. From this analysis, it was concluded that the biological function of leptins has changed significantly in the primates rlative to the function of the leptin in the common ancestor of primates and rodents.

This approach can be illustrated in a biomedically interesting family of proteins by examining the protein leptin, a protein whose mutation in mice is evidently correlated with obesity, and was previously known as the “obesity gene protein”. The protein has attracted substantial interest in the pharmaceutical industry, especially after a human gene encoding a leptin homolog was isolated. According to the conventional evolutionary paradigm, because it is a homolog of the mouse leptin, the human leptin must also play a role in obesity, and might be an appropriate target for pharmaceutical companies seeking human pharmaceuticals to combat this common condition in the first world.

DNA and protein sequences were retrieved for the genes encoding leptins. A multiple alignment for the protein sequences was constructed for the DNA sequences and the protein sequences. Congruent tress for both the DNA and protein sequences were then constructed, and sequences at the nodes of the tree reconstructed using MacClade [Mad92] and the known relationship between the organisms from which these sequences were derived. For the DNA sequences, the biologically most plausible tree proved to be the most parsimonious tree as well. The most parsimonious tree for the protein sequences proved not to be the most plausible tree (by one change) from a biological perspective. The DNA tree was taken to be definitive because of its consistency with the biological (cladistic) data.

A secondary structure prediction was made for the protein family. The evolutionary divergence of the sequences available for the leptin family is small—only 21 PAM units (point accepted mutations per 100 amino acids)—and predictions were biased to favor surface assignments [Ben94]. Thus, positions holding conserved KREND were assigned as surface residues, conserved H and Q were assigned to the surface as well, while positions holding conserved CST were assigned as uncertain.

Five separate secondary structural elements were identified. A disulfide bond was presumed to connect positions 96 and 146. These secondary structural elements can be accommodated by only a small number of overall folds. Interestingly, the pattern of secondary structure in this prediction is consistent with an overall fold that resembles that seen in cytokines such as colony stimulating factor [Hil93] and human growth hormone [deV92].

To decide whether evolutionary function may have changed under selective pressure during the divergent evolution of the protein family, silent and expressed mutations were assigned to individual branches on the evolutionary tree. For each branch of the tree, the sum of the number of silent and expressed changes were tabulated, and the ratio of expressed to silent changes calculated. These are shown in Drawing 2.

The branches on the evolutionary tree leading to the primate leptins from their ancestors at the time that rodents and primates diverged had an extremely high ratio of expressed to silent changes. From this analysis, it was concluded that the biological function of leptins has changed significantly in the primates relative to the function of the leptin in the common ancestor of primates and rodents. This conclusion has several implications of importance, not the least being for pharmaceutical companies asked whether they should explore leptins as a pharmaceutical target. At the very least, it suggests that the mouse is not a good pharmacological model for compounds to be tested for their ability to combat obesity in humans. The post-genomic analysis suggests that a primate model must be used to test those compounds, with implications for the cost of developing an anti-obesity drug based on the leptin protein.

Intriguingly, a tree can also be built for the leptin receptor. Here, the evolutionary history is not so complete. In particular, fewer primate sequences are available for the leptin receptor than for leptin itself. Thus, the reconstructed ancestral sequences are less precise with the leptin receptor family, and the assignment of expressed and silent mutations to the tree are less certain. Nevertheless, it appears that the leptin receptor has undergone an episode of rapid sequence evolution in the primate half of the family as well. The example illustrates how much sequence data is needed (much) to build reliable models of this nature, as the ambiguity in the assignment of ancestral sequences makes it possible that the receptor was evolving rapidly not only in the lineage leading to primates but also in the lineage leading to mouse.

Nevertheless, the approximate correlation between the episode of rapid sequence evolution in the leptin family and in the leptin receptor family suggests a tool that might become useful in the advanced stages of post-genomic science when evolutionary histories are very well articulated. Here, it might be possible to detect ligand-receptor relationships between protein families in the database by a correspondence between their episodes of rapid sequence evolution. Thus, ligand families should evolve rapidly (in a non-Markovian fashion) at the same time in geological history as their receptors evolve. It will be interesting to identify more sequences for primate leptin receptors to see if a more complete evolutionary history allows us to see more clearly the co-evolution of the leptin receptor and leptin itself.

### Example 5

### C. elegans Paralogs

NED distances are especially useful when comparing paralogs. Here, we need not worry so much about codon bias (it has at least been uniform among paralogs at any instant in evolutionary history). For example, we used the Master Catalog to identify all families of paralogs in the genome of C. elegans. Ca. 1250 families of paralogs with four or more members is found. We separated the families into in various classes using NED dates.
(a) Families where duplications all occurred >400 MYA
(b) Families where duplications all occurred <100 MYA
(c) Families where duplications have been ongoing throughout the past 400 MY.
(d) Families with duplications in specific episodes.

(e) Families showing a history of duplication >400 MYA, but also having more recent episodes of recruitment.

TABLE 2
presents data from just five of these 1250 families.
Number of nodes generating paralogs in indicated time

| MYA | 0-100 | 100-200 | 200-300 | 300-400 | >400 |
|---|---|---|---|---|---|
| gprod_19987 | 39 | 1 | 4 | 0 | 5 |
| Mariner transpo- | | | | | |
| sase | | | | | |
| gprod_31705 | 6 | 0 | 0 | 0 | 0 |
| similar to reverse | | | | | |
| transcriptase | | | | | |
| gprod_32709 | 11 | 3 | 0 | 0 | 1 |
| Histone H2A | | | | | |
| gprod_7894 | 5 | 2 | 0 | 0 | 2 |
| No definition line | | | | | |
| gprod_19811 | 5 | 2 | 3 | 5 | 39 |
| Serine-threonine | | | | | |
| kinase. | | | | | |

This Table immediately suggests ideas. Consider the family annotated as a serine-threonine kinase. It has 145 members in the Master Catalog; 55 or these are from elegans. The kinases generated by the recent duplications cannot part of the basic developmental plan of elegans; this was established 500 MYA. This raises questions: What is it about the serine-threonine kinases that recently diverged that might have something to do with recently evolved physiology? We then examine the Kₐ/Kₛ value within the Master Catalog trees, all with a click of a mouse button. We hypothesize which descendants of recent duplications performing the derived function, and which perform the primitive function. Dating the divergence, we try to make statements about changes in nematode biology that might be associated with the duplication. These hypotheses can now be tested by experiment (knock-outs, in particular).

One observation apparent from the Table is that genes that have multiple recent recruitments in C. elegans are unlikely to have clearly identifiable homologs in other phyla, while those that have few recent recruitments are more likely than average to have clearly identifiable homologs in other phyla.

### REFERENCES

[Akh97] Akhtar, M., LeeRobichaud, P., Akhtar, M. E., Wright, J. N. (1997) The impact of aromatase mechanism on other P450s. J. Steroid Biochem. Mol. Biol. 61, 127-132.
[Alt87a] Altschuh, D., Lesk, A. M., Bloomer, A. C., Klug, A. (1987a) Correlation of coordinated amino acid substitutions with function in tobacco mosaic-viruses, Prot. Engng. 1, 228-236.
[Alt87b] Altschuh, D., Lesk, A. M., Bloomer, A. C., Klug, A. (1987b) Correlation of coordinated amino acid substitutions with function in viruses related to tobacco mosaic-virus, J. Mol. Biol. 193, 693-707.
[Ant99] Antunes, M. T., Cahuzac, B. (1999) Crocodilian faunal renewal in the Upper Oligocene of Western Europe. Comptes Rend. L'Acad. Sci. Serie II Fascicule A-Sci. Terre Planetes. 328, 67-72.
[Aza93] Azanza, B. (1993) Systematics and evolution of the genus Procervulus (Cervidae, Artiodactyla, Mammalia) of the lower Miocene of Europe. Comptes Rend. L'Acad. Sci. Serie II. 316, 717-723.
[Bal93] Baldwin, E. P., Hajiseyedjavadi, W. A. and Matthews, B. W. (1993) The role of backbone flexibility in the accomodation of variants that repack the core of T4 lysozyme. Science 262, 1715-1718.
[Bat00] Bateman, A., Birney, E., Durbin, R., Eddy, S. R., Howe, K. L., Sonnhammer, E. L. L. (2000) The Pfam protein families database. Nucl. Acids Res. 28, 263-266.
[Ben88] Benner, S. A., Ellington, A. D. Interpreting the behavior of enzymes. Purpose or pedigree? CRC Crit. Rev. Biochem. 23, 369-426 (1988).
[Ben89a] Benner, S. A. (1989) Patterns of divergence in homologous proteins as indicators of tertiary and quaternary Structure. Adv. Enzym. Regulation 28, 219-236.
[Ben91] Benner, S. A., Gerloff, D. L. (1991) Patterns of divergence in homologous proteins as indicators of secondary and tertiary structure. The catalytic domain of protein kinases. Adv. Enzyme Regulat. 31, 121-181.
[Ben94] Benner, S. A., Badcoe, I., Cohen, M. A., Gerloff, D. L. Bonafide prediction of aspects of protein conformation. Assigning interior and surface residues from patterns of variation and conservation in homologous protein sequences. J. Mol. Biol. 235, 926-958 (1994).
[Ben97] Benner, S. A., Cannarozzi, G., Chelvanayagam, G., Turcotte, M. (1997) Bona fide predictions of protein secondary structure using transparent analyses of multiple sequence alignments. Chem. Rev. 97, 2725-2843.
[Ben98] Benner, S. A., Trabesinger-Ruef, N., Schreiber, D. R. (1998) Exobiology and post-genomic science. Converting primary structure into physiological function. Adv. Enzyme Regul. 38, 155-180.
[Boe97] Boerboom, D., Kerban, A., Sirois, J. (1997) Molecular characterization of the equine cytochrome P450 aromatase cDNA and its regulation in preovulatory follicles. Biol. Reprod. 56, 479479, Suppl. 1.
[Bor90] Bordo, D., Argos, P. (1990) Evolution in protein cores. Constraints in point mutations as observed in globin tertiary structures, J. Mol. Biol. 211, 975-988.
[Buc88] Buck, C. D. (1988) A Dictionary of Selected Synonyms in the Principal European Languages. Chicago, University of Chicago Press, Paperback ed., p. 160.
[Cal84] Callard, G. V., Pudney, J. A., Kendall, S. L., Reinboth, R. (1984) In vitro conversion of androgen to estrogen in Amphioxus gonadal tissues. Gen. Comp. Endocrinol. 56, 53-58.
[Cal97] Callard, G. V., Tchoudakova, A. (1997) Evolutionary and functional significance of two CYP19 genes differentially expressed in brain and ovary of goldfish. J. Steroid Biochem. Mol. Biol. 61, 387-392.
[Car88] Carroll, R. L. (1988) Vertebrate Paleontology and Evolution. N.Y., Freeman.
[Cha97] Chang, X. T., Kobayashi, T., Kajiura, H., Nakamura, M., Nagahama, Y. (1997) Isolation and characterization of the cDNA encoding the tilapia (Oreochromis niloticus) cytochrome P450 aromatase (P450arom), Changes in P450arom mRNA, protein and enzyme activity in ovarian follicles during oogenesis. J. Mol. Endocrinol. 18, 57-66.
[Che97] Chelvanayagam, G., Eggenschwiler, A., Knecht, L., Gonnet, G. H., Benner, S. A. An analysis of simultaneous variation in protein structures. Protein Engineering 10, 307-316 (1997).
[Che98] Chelvanayagam, G., Knecht, L., Jenny, T. F., Benner, S. A. Gonnet, G. H. A combinatorial distance constraint approach to predicting protein tertiary models from known secondary structure. Fold. Design 3, 149-160 (1998).
[Cho82] Chothia C., Lesk, A. M. (1982) Evolution of proteins formed by b-sheets I. Plastocyanin and azurin, J. Mol. Biol., 160, 309-323.
[Cho96] Choi, I., Simmen, R. C. M., Simmen, F. A. (1996) Molecular cloning of cytochrome P450 aromatase complementary deoxyribonucleic acid from periimplantation porcine and equine blastocysts identifies multiple novel 5′-untranslated exons expressed in embryos, endometrium, and placenta. Endocrinol. 137, 1457-1467.
[Cho97a] Choi, I., Collante, W. R., Simmen, R. C. M., Simmen, F. A. (1997a) A developmental switch in expression from blastocyst to endometrial/placental-type cytochrome p450 aromatase genes in the pig and horse. Biol. Reprod. 56, 688-696.
[Cho97b] Choi, I. H., Troyer, D. L., Cornwell, D. L., Kirby-Dobbels, K. R., Collante, W. R., Simmen, F. A. (1997b) Closely related genes encode developmental and tissue isoforms of porcine cytochrome P450 aromatase. DNA Cell. Biol. 16,769-777.
[Col41] Colbert, E. H. (1941) The osteology and relationships of Archaeomeryx, an ancestral ruminant. Amer. Mus. Novit. 1135, 1-24.
[Con97] Conley, A., Corbin, J., Smith, T., Hinshelwood, M., Liu, Z., Simpson, E. (1997) Porcine aromatases, studies on tissue-specific functionally distinct isozymes from a single gene? J. Steroid Biochem. Mol. Biol. 61, 407-413.
[Con96] Conley, A. J., Corbin, C. J., Hinshelwood, M. M., Liu, Z., Simpson, E. R., Ford, J. J., Harada, N. (1996) Functional aromatase expression in porcine adrenal gland and testis. Biol Reprod. 54,497-505.
[Coo78] Cooke, H. B. S., Wilkinson, A. F. (1978) Suidae and Tayassuidae, in Evolution of African Mammals, V. J. Maglio and H. B. S. Cooke, eds. Cambridge, Harvard University Press, 438-482.
[Cor00] Corpet, F., Servant, F., Gouzy, J., Kahn, D. (2000) ProDom and ProDom-CG: Tools for protein domain analysis and whole genome comparisons. Nucl. Acids Res. 28, 267-269.
[Del96] Delarue, B., Mittre, H., Feral, C., Benhaim, A., Leymarie, P. (1996) Rapid sequencing of rabbit aromatase cDNA using RACE PCR. Comptes Rend. L'Acad. Sci. Serie III Sciences De La Vie-Life Sciences 319, 663-670.
[Del98] Delarue, B., Breard, E., Mittre, H., Leymarie, P. (1998) Expression of two aromatase cDNAs in various rabbit tissues. J. Steroid Biochem. Mol. Biol. 64, 113-119.
[deV92] de Vos, A. M., Ultsch, M. & Kossiakoff, A. A. (1992). Human growth-hormone and extracellular domain of its receptor. Crystal-structure of the complex. Science 255, 306-312].
[Dos73] Dostal, J., Matousek, J. (1973) Isolation and some chemical properties of aspermatogenic substance from bull seminal vesicle fluid. J. Reprod. Fertil. 33, 263-274.
[Eis81] Eisenberg, J. F. (1981) The Mammalian Radiations. An Analysis of Trends in Evolution, Adaptation, and Behavior. Chicago, Univ. Chicago Press, p 196.
[Fit71] Fitch, W. (1971) Towards defining the course of evolution. Minimum change for a specific tree topology. Syst. Zoology 20, 406-416.
[For96] Fortelius, M., van der Made, J., Bemor, R. L. (1996) Middle and Late Miocene Suoidea of Central Europe and the Eastern Mediterranea, Evolution, Biogeography and Paleoecology. in The Evolution of Western Eurasian Neogene Mammal Fanas. R. L. Bernor, V. Fahlbusch, and H.-W. Mittmann eds. Columbia Univ. Press, 348-377.
[Für95] Fürbab R, Vanselow J. (1995) An aromatase pseudogene is transcribed in the bovine placenta. Gene 154,287-291.
[Gob94] Göbel, U, Sander, C. Schneider, R. and Valencia, A (1994) Correlated mutations and residue contacts in proteins. Proteins: Struc. Funct., Gen. 18, 309-317.
[Goh00] Goh, C.-S., Bogan, A. A., Joachimiak, M., Walther, D., Cohen, F. W. (2000) Co-evolution of Proteins with their Interaction Partners. J. Mol. Biol. 299, 283-293.
[Gon90] Gonzalez, F. J., Nebert, D. W. (1990) Evolution of the P450-gene superfamily. Animal plant warfare, molecular drive and human genetic-differences in drug oxidation. Trends Genet. 6, 182-186.
[Gon92] Gonnet, G. H., Cohen, M. A., Benner, S. A. (1992) Exhaustive matching of the entire protein sequence database. Science 256, 1443-1445.
[Gon91] Gonnet, G. H., Benner, S. A. (1991) Computational Biochemistry Research at ETH. Technical Report 154, Departement Informatik, March, 1991.
[Har88] Harada, N. (1988) Cloning of a complete cDNA encoding human aromatase, immunochemical identification and sequence analysis. Biochem. Biophys. Res. Comm. 156, 725-732.
[Hic90] Hickey, G. J., Krasnow, J. S., Beattie, W. G., Richards, J. S. (1990) Aromatase cytochrome P450 in rat ovarian granulosa cells before and after luteinization. Adenosine 3′,5′-monophosphate-dependent and independent regulation. Cloning and sequencing of rat aromatase cDNA and 5′ genomic DNA. Mol. Endocrinol. 4, 3-12.
[Hil93] Hill, C., P., Osslund, T. D., Eisenberg, D. (1993) The structure of granulocyte colony stimulating factor and its relationship to other growth factors. Proc. Nat. Acad. Sci. 90, 5176-5181.
[Hin93] Hinshelwood, M. M., Corbin, C. J., Tsang, P. C. and Simpson, E. R. (1993) Isolation and characterization of a complementary deoxyribonucleic acid insert encoding bovine aromatase cytochrome P450. Endocrinology 133, 1971-1977.
[Jer95] T. M. Jermann, J. G. Opitz, J. Stackhouse, J. and S. A. Benner, Reconstructing the evolutionary history of the artiodactyl ribo nuclease superfamily. Nature 374, 57-59 (1995).
[Jol89] Jolles, J., Jolles, P., Bowman, B. H., Prager, E. M., Stewart, C. B., & Wilson, A. C. (1989) J. Mol. Evol. 28, 528-535.
[Juk69] Jukes, T. H., Cantor, C. R. (1969) Evolution of proteins molecules. in Mammalian Protein Metabolism, H. N. Munro, ed. N. Y. Academic Press, pp. 21-123.
[Kim80] Kimura, M. (1980) A simple method for estimating evolutionary rates of base substitution through comparative studies of nucleotide sequences. J. Mol. Evol. 16, 111-120.
[Kni91] Knighton, D. R., Zheng, J., Ten Eyck, L., Ashford, F. V. A., Xuong, N. H., Taylor, S. S., Sowadski, J. M. (1991) Crystal structure of the catalytic subunit of cyclic adenosine-monophosphate dependent protein-kinase. Science 253, 407-414.
[Kre95] Kreitman, M., Akashi, H. Ann. Rev. Ecol. Syst. 26, 403-422 (1995).
[Les80] Lesk, A. M., Chothia, C. (1980) How different amino acid sequences determine similar protein structures. The structure and evolutionary dynamics of the globins. J. Mol. Biol. 136, 225-270.
[Les82] Lesk, A. M., Chothia, C. (1982) Evolution of proteins formed by b-sheets II. The core of the immunoglobulin domains, J. Mol. Biol., 160, 325-342.
[Li85] Li, W. H., Wu, C. I., Luo, C. C. (1985) A new method for estimating synonymous and nonsynonymous rates of nucleotide substitution considering the relative likelihood of nucleotide and codon changes. Mol. Biol. Evol. 2, 150-174.
[Li97] Li, W.-H. Molecular Evolution (Sinauer Assc., Inc., Sunderland, Mass., 1997).
[Lic96] 0. Lichtarge, H. R. Bourne, F. E. Cohen, An evolutionary trace analysis defines binding surfaces common to protein families. J. Mol. Biol. 257, 342-358 (1996).
[Lim89] Lim, W. A., Sauer, R. T. (1989) Alternative packing arrangements in the hydrophobic core of 1-repressor, Nature (London) 399, 31-36.
[Lim92] Lim, W. A., Farruggio, D. C., Sauer, R. T. (1992) Structural and energetic consequences of disrupting mutations in a protein core. Biochemistry 31, 4324-4333.
[Mad92] W. P. Maddison, D. R. Maddison, MacClade. Analysis of Phylogeny and Character Evolution. Sinauer Associates, Sunderland Mass. (1992).
[Mar99] Marcotte, E. M., M. Pellegrini, H. L. Ng, D. W. Rice, T. O. Yeates, and D. Eisenberg (1999) Detecting protein function and protein-protein interactions from genome sequences. Science 285, 751-753.
[Mat73] Matousek, J. (1973) The effect of bovine seminal ribonuclease on cells of Crocker tumor in mice. Experientia 29, 858.
[McD91] McDonald, J. H., Kreitman, M. (1991) Adaptive protein evolution at the adh locus in Drosophil Nature 351, 652-654.
[McP88] McPhaul, M. J., Noble, J. F., Simpson, E. R., Mendelson, C. R., Wilson, J. D. (1988) The expression of a functional cDNA encoding the chicken cytochrome P-450-arom (aromatase) that catalyzes the formation of estrogen from androgen. J. Biol. Chem. 263, 16358-16363.
[Mes97] Messier, W., Stewart, C. B. (1997) Episodic adaptive evolution of primate lysozymes (1997) Nature 385,151-154.
[Miy95] Miyamoto, M. M., Fitch, W. M. (1995) Testing the covarion hypothesis of molecular evolution. Mol. Biol. Evol. 12, 503-513.
[Mur99] Murelaga, X., de Broin, F. D., Suberbiola, X. P., Astibia, H. (1999) Two new chelonian species from the Lower Miocene of the Ebro Basin (Bardenas Reales of Navarre). Comptes Rend. L'Acad. Sci. Serie II Fascicule A-Sci. Terre Planetes. 328, 423-429.
[Neb91] Nebert, D. W., Nelson, D. R., Coon, M. J., Estabrook, R. W., Feyereisen, R., Fujiikuriyama, Y., Gonzalez, F. J., Guengerich, F. P., Gunsalus, I. C., Johnson, E. F., Loper, J. C., Sato, R., Waterman, M. R., Waxman, D. J. (1991) The P450 superfamily. Update on new sequences, gene-mapping, and recommended nomenclature. DNA Cell Biol. 10,1-14.
[Neh94] Neher, E. (1994) How frequent are correlated changes in families of protein sequences? Proc. Natl. Acad. Sci. U.S.A. 91,98-102.
[Nei86] Nei, M., Gojobori, T. (1986) Simple methods for estimating the numbers of synonymous and nonsynonymous nucleotide substitutions. Mol. Biol. Evol. 3, 418-426.
[Oos86] Oosawa, K., Simon, M. (1986) Analysis of mutations in the transmembrane region of the aspartate chemoreceptor in Escherichia coli. Proc. Nat. Acad. Sci. 83, 6930-6934.
[Pam93] Pamilo P, Bianchi, N. O. (1993) Evolution of the zfx and zfy genes—rates and interdependence between the genes. Mol. Biol. Evol. 1, 271-281.
[Pel99] Pellegrini, M., Marcotte, E. M., Thompson, M. J., Eisenberg, D., Yeates, T. O. Assigning protein functions by comparative genome analysis: Protein phylogenetic profiles PNAS 96, 4285-4288 1999.
[Pil41] Pilgrim, G. E. (1941) The dispersal of the Artiodactyla, Biol. Rev., 16, 134-163.
[Pro94] Prothero, D. R. (1994) The Eocene-Oligocene Transition, Paradise Lost NY, Columbia Univ. Press.
[Qi98] Qi, T., Beard, K. C. (1998) Late Eocene sivaladapid primate from Guangxi Zhuang Autonomous Region, People's Republic of China. J. Human Evol. 35, 211-220.
[Roc96] Rocek, Z. (1996) The salamander Brachycormus noachicus from the Oligocene of Europe, and the role of neoteny in the evolution of salamanders. Palaeontology 39, 477-495.
[Ros82] Rose, K. D. (1982) Skeleton of Diacodexis, oldest known artiodactyl. Science 236, 621-623.
[Sav86] Savage, R. J. G., Long M. R. (1986) Mammal Evolution. An Illustrated Guide. N.Y., Facts on File Publ., p 213.
[Sco37] Scott, W. B. (1937) A History of Land Mammals in the Western Hemisphere. N.Y. McMillan.
[She94] Shen, P., Campagnoni, C. W., Kampf, K., Schlinger, B. A., Arnold, A. P., Campagnoni, A. T. (1994) Isolation and characterization of a zebra finch aromatase cDNA. In situ hybridization reveals high aromatase expression in brain. Brain Res. Mol. Brain Res. 24, 227-237.
[Shi94] Shindyalov, I. N., Kolchanov, N. A. and Sander, C. (1994) Can three-dimensional contacts in protein structures be predicted by analysis of correlated mutations? Prot. Engng. 7, 349-358.
[Sim97] Simpson, E. R., Michael, M. D., Agarwal, V. R., Hinshelwood, M. M., Bulun, S. E., Zhao, Y. (1997) Expression of the CYP19 (aromatase) gene. An unusual case of alternative promoter usage. FASEB J., 11, 29-36.
[Sou81] Soucek, J., Matousek, J. (1981) Inhibitory effect of bovine seminal ribonuclease on activated lymphocytes and lymphoblastoid cell lines in vitro. Folia Biol. Praha 27, 334-345.
[Sou83] Soucek, J., Hrubá, A., Paluska, E., Chudomel, V., Dostál, J., Matousek, J. (1983) Immunosuppressive effects of bovine seminal fluid fractions with ribonuclease activity. Folia biologica (Praha) 29, 250-261.
[Sou86] Soucek, J., Chudomel, V., Potmesilova, I., Novak, J. T. (1986) Effect of ribonucleases on cell, mediated lympholysis reaction and on GM, CFC colonies in bone marrow culture. Nat. Immun. Cell Growth Regul. 5, 250-258.
[Ste87] Stewart, C. B., Schilling, J. W., Wilson, A. C. (1987) Adaptive evolution in the stomach lysozymes of foregut fermenters. Nature 330, 401-404.
[Str00] Strickberger, M. W. (2000) Molecular Evolution, Sudbury Mass., Jones and Bartlett (p. 644).
[Stu90] Stucky, R. K. (1990) Evolution of land mammal diversity in North America during the Cenozoic. Curr. Mammalogy 2, 375-432.
[Swo96] Swofford, D. L., Olsen, G. J., Waddell, P. J., & Hillis, D. M. (1996) Phylogenetic Inference in Molecular Systematics (eds. Hillis, D. M., Moritz, C. & Mable, B. K.) 407-514 (Sinauer Assc., Inc., Sunderland, Mass., 1996).
[Tan95] Tanaka, M., Fukada, S., Matsuyama, M., Nagahama, Y. (1995) Structure and promoter analysis of the cytochrome P450 aromatase gene of the teleost fish, medaka (Oryzias latipes). J. Biochem. 117, 719-725.
[Tay94] Taylor, W. R. and Hatrick, K. (1994) Compensating changes in protein multiple sequence alignments, Prot. Engng. 7: 341-348.
[Tch92] Tchernov, E. (1992) The Afro-Arabian component in the levantine mammalian fauna. A short biogeographical review. Israel J. Zoology 38, (3-4) 155-192.
[Ter91] Terashima, M., Toda, K., Kawamoto, T., Kuribayashi, I., Ogawa, Y., Maeda, T., Shizuta, Y. (1991) Isolation of a full-length cDNA encoding mouse aromatase P450. Arch. Biochem. Biophys. 285, 231-237.
[Tra94] Trant, J. M. (1994) Isolation and characterization of the cDNA encoding the channel catfish (Ictalurus punctatus) form of cytochrome P450arom. Gen. Comp. Endocrinol. 95, 155-168.
[Tra96] Trabesinger-Ruef, N., Jermann, T. M., Zankel, T. R., Durrant, B., Frank, G., Benner, S. A. (1996) Pseudogenes in ribonuclease evolution. A source of new biomacromolecular function? FEBS Lett. 382, 319-322.
[van99] van der Made J, Tuna V. (1999) A tetraconodontine pig from the Upper Miocene of Turkey. Trans. Royal Soc. Edinburgh. Earth Sci. 89, 227-230.
[Ves80] Vescia, S., Tramontano, D., Augusti-Tocco, G., D'Alessio, G. (1980) In vitro studies on selective inhibition of tumor cell growth by seminal ribonuclease. Cancer Res. 40, 3740.
[Wol78] Wolfe, J. A. (1978) A paleobotanical interpretation of Tertiary climates in the Northern Hemisphere. American Sci. 66, 694-703.
[Yan97] Yang, Z. H. PAML: a program package for phylogenetic analysis by maximum likelihood. Comput. Appl. Biosci. 13, 555-556 (1997).

1. A method for predicting the secondary structure of proteins comprising (a) obtaining a multiplicity of homologous protein sequences, (b) constructing an alignment of the multiplicity of sequences, and (c) analyzing patterns of conservation and variation at sites in the multiple sequence alignment, wherein said multiplicity comprises at least 16 homologous protein sequences.

2. The method of claim 1, wherein said set comprises at least eight pairs of proteins, wherein the proteins in each pair are at least 80% identical in sequence.

3. The method of claim 1, wherein said analysis incorporates a model for the evolutionary divergence of said homologous protein sequences.

4. A method for the identification of a secondary structural element that may be involved in functional adaptation, wherein said method comprises (a) obtaining a multiplicity of homologous protein sequences and their encoding DNA sequences, (b) constructing an alignment of the multiplicity of sequences, (c) constructing an evolutionary tree that models the evolutionary history of the family of genes and proteins represented by said sequences, (d) constructing models of the sequences of the genes and their encoded proteins at nodes in the tree, (e) assigning changes in the gene and protein sequences to lines connecting such nodes, and (f) calculating the ratio of non-synonymous to synonymous nucleotide substitutions for said lines at sites in said alignment that are part of said element, wherein said secondary structural element is identified as possibly being involved in functional adaptation if the said ratio is in excess of a preselected value.

5. A method for identifying a pair of proteins that may come into physical contact when they function comprising (a) obtaining a multiplicity of homologous protein sequences and their encoding DNA sequences that are related to each member of the pair, (b) constructing an alignment of the multiplicity of sequences, (c) constructing an evolutionary tree that models the evolutionary history of the family of genes and proteins represented by said sequences, (d) constructing models of the sequences of the genes and their encoded proteins at nodes in the tree, and (e) assigning events in the gene and protein sequences to lines connecting such nodes, wherein said pair of proteins is identified as possibly coming into physical contact when they function if events assigned to a line in one family correlate with events assigned to lines representing contemporaneous episodes in the other family.

6. The method of claim 5, wherein said events comprise episodes of sequence evolution associated with a ratio of non-synonymous to synonymous nucleotide substitutions in excess of a preselected value.

7. The method of claim 5 wherein one protein in said pair is a peptide hormone, and the other protein in said pair is a peptide hormone receptor.

8. A method for estimating the date since a pair of proteins diverged comprising aligning the sequences of said pair, identifying in said alignment each cysteine, aspartic acid, glutamic acid, phenylalanine, histidine, lysine, asparagine, glutamine, and tyrosine that is conserved in the pair, totalling the number of these, and summing the number of these wherein the respective codon is conserved, obtaining a ratio by dividing said sum by said total, subtracting 0.5 from said ratio, multiplying the difference by 2, taking the natural logarithm of the product and dividing by a number that is the estimate for the first order rate constant for replacement at the silent sites in said codons.

9. A method for identifying a protein family that may be associated with a change in a physiology in a taxon, said method comprising (a) obtaining a multiplicity of homologous protein sequences for said family, (b) constructing an alignment of the multiplicity of sequences, (c) constructing an evolutionary tree that models the evolutionary history of said family, and (d) correlating events in said family in time with the change in said physiology.

10. The method of claim 9, wherein said time is estimated using the paleontological record.

11. The method of claim 9, when dating events in the evolutionary history of said family is done using the method of claim 8.

12. The method of claim 9, wherein said events comprise gene duplications.
